(19) United States
(12) Patent Application Publication
(10) Pub. No.: US 2011/0118196 A1
(43) Pub. Date: May 19, 2011

(54) ANTI-PHTHIRAPTERA TREATMENT COMPOSITIONS

(75) Inventor: Ahron Chazot, Kibbutz Maagan - Doar-Na Emek HaYarden (IL)

(73) Assignee: Lice Up Ltd., Petach-Tikva (IL)

(21) Appl. No.: 13/002,768

(22) PCT Filed: Jul. 6, 2009
(86) PCT No.: PCT/IL2009/000674

§ 371 Date: Jan. 6, 2011

Related U.S. Application Data
(60) Provisional application No. 61/078,399, filed on Jul. 6, 2008.

(51) Int. Cl. A61K 31/7004; A61P 33/14

(52) U.S. Cl. 514/23

(57) ABSTRACT

Liquid ovicidal/pediculicidal compositions for eradication of ectoparasites, such as head-lice and other phthiraptera, are disclosed. The compositions comprise 5-50 weight percents of a film-forming carbohydrate and optionally 1-10 weight percents of oil, and are washable within a time period of 1-5 minutes from hair at a length of less than 60 cm in a stream of water of at least 5 liters per minute and a temperature lower than 40° C. Hair care formulations such as shampoos, conditioners, mousses, gels and the likes containing such an ovicidal/pediculicidal composition are further disclosed. Also disclosed are methods preparing and using the compositions and formulations.

### FIELD AND BACKGROUND OF THE INVENTION

The present invention, in some embodiments thereof, relates to the field of cosmetics and household remedies for eradication of ectoparasites, and more particularly, but not exclusively, to anti-phthiraptera treatment compositions and methods using the same.

Infestation of the body by lice and other phthiraptera and parasitic insects is an age old problem, and reference to these pests can be found throughout documented human history. Lice have been responsible for the spread of typhus, causing decimation of armies and navies of the military powers of the 15^{th} to 18^{th} centuries. Lice are still considered as disease vectors and present serious health problems throughout the world; not only do lice carry a wide variety of bacteria on their exterior surfaces, but their fecal matter transmits disease when it enters the puncture wounds lice inflict during feeding.

The human lice genus includes pubic lice, body lice and head lice, each having specific characteristics with regard to habitat and feeding. The most common, namely the head lice, are small hard-shelled ectoparasites which cling to hair follicles while feeding, mating and laying eggs. The louse must remain on the head as it will die within a short period of time when removed. Ideal conditions include an adequate food supply, environmental temperatures from about 28° C. to about 32° C., and relative humidity from about 70% to about 90%. Poor hygienic and grooming habits are also known to contribute significantly to the spread of lice, thus, lice infestations are most serious in tropical areas where the inhabitants have both substandard hygienic facilities and practices.

The louse's hard chitinous shell serves as protection from external elements. Lice eggs (ova) are similarly protected by a chitinous sheath surrounding the eggs and attached to the hair follicles. Although the lice may be affected by the use of an insecticide, often the ova remain resistant to attack. Thus, the optimum treatment should kill the nymph and adult lice and interrupt the gestation of the ova.

Numerous preparations and treatment methods are known for treating lice infected areas, utilizing natural or synthetic chemical pesticides and/or repellants as pediculicides. Most chemical treatments suffer from low efficiency in treating lice that have survived a first treatment and which have developed resistance thereto, leading to increase in dosages which may result in human health risks. Chemical treatments may also be ineffective in destroying lice eggs. Manual hair combing is commonly performed to destroy lice eggs, however it is tedious, and due to the large number of eggs, it is also ineffective.

Biologically-active agents for the control of lice are well known in the art. Lindane (gamma-benzene hexachloride), synergized natural pyrethrins, and synthetic derived compounds known as pyrethroids have all been used as pediculicides in lice treatment compositions. However, since lindane has a poor safety profile and lice have developed a significant degree of resistance to it, natural pyrethrins and synthetic pyrethroids are routinely chosen for use in pediculicide and ovicide compositions.

Natural pyrethrins are made from extracts of naturally insecticidal chrysanthemum flowers and have been used since the early 1930's. European Patent Application 191,236; European Patent Application 262,885; and British Patent Specification 1,593,601, all disclose the use of natural pyrethrins for treating lice. U.S. Pat. No. 4,668,666, teaches that natural pyrethrins' poor environmental stability is a severe drawback in treatment of lice, as its low residual action, due to this instability, necessitates frequent follow-up treatments.

Synthetic pyrethroids became popular during World War II when chrysanthemum flowers became scarce. Besides being cheaper and more readily available, these agents were also somewhat more stable than the natural product. This long-term stability extended toxicity to future hatching ova. Although more effective against lice than natural pediculicides in general, some of the synthetic actives are more toxic to the subject being treated.

Other anti-lice formulations eliminating insecticides are known in the art. Alkanols are known to effectively control head lice infestations. British Patent Specification 1,604,857 and European Application 262,885, disclose the use of alkanols, either singularly or as adjuvants, as anti-lice agents. Primary as well as aromatic alkanols are disclosed therein as components of anti-lice compositions, such as sprays and shampoos.

Cationic surfactants are known to synergize pediculicides, as disclosed in British Patent Specification 1,593,601. U.S. Pat. No. 4,183,913 specifically discloses the use of quaternary ammonium salts to synergize pediculicides. Quaternary ammonium salts are disclosed as a stand-alone pediculicide for head lice in European Patent Application 191,236.

U.S. Pat. No. 5,518,736 discloses a method of preparing natural-oil-containing emulsions and microcapsules, and their uses for fighting head lice, by teaching a sustained-release biodegradable lice repelling preparation comprising, in the form of an aqueous emulsion, at least one natural oil, an anionic emulsifier and chitosan.

U.S. Patent Application having Publication No. 20040259836 teaches dry powdery or granular compositions containing saccharides and fine hydrophobic fumed silica (fused quartz) solid and insoluble particles. This document fails to teach effective application of these dry compositions, and demonstrates methods of use including having the composition be applied on the parasite by gentle stroking.

### SUMMARY OF THE INVENTION

The present invention, in some embodiments thereof, relates to a composition for eradication of ectoparasites, and more particularly, but not exclusively, to anti-phthiraptera treatment compositions, hair care formulations containing same and methods utilizing the same. In some embodiments, the compositions and hair care formulations are based on an emulsion of a film-forming carbohydrate and oil which practically kills lice and other insects including their ova, and in the case of ectoparasites that live on human hair, loosens the grip of the insects to the hair, thereby making the mechanical removal thereof much more effective.

According to one aspect of embodiments of the present invention there is provided a hair care formulation comprising from about 20 weight percents to about 60 weight percents of a liquid ovicidal/pediculicidal composition, the liquid ovicidal/pediculicidal composition comprising: a film-forming carbohydrate in an amount that ranges from about 5 weight percents to about 50 weight percents; and water.

According to some embodiments of the invention, the hair care formulation being identified for eradicating a population of at least one ectoparasite.

According to some embodiments of the invention, the formulation is being washable within a time period of less than 5 minutes from hair at a length of 5 cm under water shower stream of 20 liters per minutes and a water temperature of 37° C.

According to some embodiments of the invention, the formulation is washable within a time period of less than 1 minute from hair at a length of 5 cm under water shower stream of 20 liters per minutes and a water temperature of 37° C.

According to some embodiments of the invention, the composition further comprises oil in an amount that ranges from about 1 weight percent to about 20 weight percents of the total weight of the composition.

According to some embodiments of the invention, the composition further comprises an emulsifier in an amount that ranges from about 0.5 weight percent to about 5 weight percents of the total weight of the composition.

According to some embodiments of the invention, the formulation is in a form selected from the group consisting of a shampoo, a conditioner, a mask, a mousse, a gel, a hairspray, a lotion, a moisturizer, a tonic and a serum.

According to some embodiments of the invention, the ectoparasite is selected from the group consisting of Pediculus humanus capitis (head lice), Pediculus humanus corporis, Phthirus pubis, Cimicidae Cimex lectularius and Sarcoptes scabiei.

According to some embodiments of the invention, the ectoparasite is Pediculus humanus capitis.

According to some embodiments of the invention, the film-forming carbohydrate is selected from the group consisting of a monosaccharide, a disaccharide, a polysaccharide, a purified saccharide, an impure saccharide, a crude saccharide, a mixture of saccharides, molasses, syrup, treacle and any combination thereof.

According to some embodiments of the invention, the film-forming carbohydrate comprises at least one substance selected from the group consisting of allose, altrose, arabinose, a beet-sugar syrup, a cane-sugar syrup, a corn syrup, cellobiose, deoxyribose, dextrose, erythrose, erythrulose, fructose, fructose syrup, fucose, fructan, galactitol, galactosamine, galactose, gentiobiose, gluconic acid, glucosamine, glucose, glucose syrup, glucuronic acid, glycerol (glycerine), gulose, a high fructose starch-based syrup (HFSS), a high-fructose corn syrup (HFCS), idose, inositol, isomalt, isoglucose, isomaltose, isomaltulose/palatinose, lactitol, lactose, lactulose, levulose, lyxose, maltitol, maltose, maltodextrin, maltotriose, mannitol, mannosamine, mannose, melibiose, nystose, propylene glycol, psicose, raffinose, rhamnose, ribose, ribulose, saccharose, sorbitol, sorbose, sucrose, tagatose, talose, threose, trehalose, turanose, xylitol, xylobiose, xylose, xylulose, α,β-trehalose (neotrehalose), β,β-trehalose (isotrehalose), β-allopyranose, and mixtures and syrups thereof.

According to some embodiments of the invention, the film-forming carbohydrate is glucose syrup having a dextrose equivalent value (DE) lower than 55.

According to some embodiments of the invention, the film-forming carbohydrate is glucose syrup having a dextrose equivalent value (DE) lower than 45.

According to some embodiments of the invention, the film-forming carbohydrate is glucose syrup having a dextrose equivalent value (DE) of 42.

According to some embodiments of the invention, the oil is selected from the group consisting of acai oil, agarwood oil, algae oil, allspice oil, almond oil, amaranth oil, amur cork tree fruit oil, anise oil, apple seed oil, apricot oil, argan oil, artichoke oil, avocado oil, babassu oil, balanos oil, basil oil, bay leaf oil, ben (moring a oleifera) oil, bergamot oil, blackcurrant seed oil, bladderpod oil, borage seed oil, borneo tallow nut oil, bottle gourd oil, brucea javanica oil, buffalo gourd oil, burdock (bur) oil, camphor oil, candlenut (kukui nut) oil, cannabis oil, canola oil, cape chestnut (yangu) oil, carob pod oil, carrot seed oil, cashew oil, cassia oil, castor oil, cedar oil, celery oil, chamomile oil, chaulmoogra oil, cinnamon oil, clary sage oil, clove oil, cocklebur oil, cocoa oil, coconut oil, coconut oil, cohune oil, copaiba oil, copra oil, coriander seed oil, corn oil, cottonseed oil, crambe oil, cumin oil, cuphea oil, dammar oil, dika oil, eucalyptus oil, evening primrose oil, false flax (camelina sativa) oil, flax seed oil, frankincense oil, galangal oil, ginger oil, grape seed oil, grapefruit oil, hazelnut oil, hemp oil, honge oil, hops oil, hyssop oil, jasmine oil, jatropha oil, jojoba oil, juniper oil, kapok seed oil, lallemantia oil, lavender oil, lemon grass oil, lemon oil, lime oil, linseed oil, macadamia oil, mango oil, manuka oil, marjoram oil, manila oil, meadowfoam seed oil, melaleuca oil, mongongo nut oil, mowrah oil, mustard oil, mustard oil, myrrh oil, neem oil, nutmeg oil, nutmeg oil, ojon oil, okra seed oil, olive oil, orange oil, oregano oil, palm oil, papaya seed oil, patchouli oil, peanut oil, pecan oil, peppermint oil, pequi oil, perilla seed oil, pine nut oil, pine oil, pistachio oil, poppy seed oil, prune kernel oil, pumpkin seed oil, quinoa oil, radish oil, ramtil oil, rapeseed oil, rice bran oil, rose hip seed oil, rose oil, rosemary oil, rosewood oil, royle oil, sacha inchi oil, safflower oil, sage oil, salicornia oil, sandalwood oil, sassafras oil, scented geranium oil, sea buckthorn oil, sesame oil, shea oil, snowball seed oil, soybean oil, spearmint oil, stillingia oil, sunflower oil, tall oil, tamanu oil, tangerine oil, tea seed oil, tea tree oil, thistle oil, thyme oil, tocopherol (vitamin E), tocopheryl acetate, tomato seed oil, tonka bean (cumaru) oil, tung oil, valerian oil, vernonia oil, walnut oil, watermelon seed oil, wheat germ oil, wintergreen oil, ylang-ylang oil and any combination thereof.

According to some embodiments of the invention, the emulsifier is selected from the group consisting of cationic emulsifier, an anionic emulsifier, a non-ionic emulsifier and combinations thereof.

According to some embodiments of the invention, the emulsifier is selected from the group consisting of sodium dodecyl sulfate, a phospholipid, a glycolipid, a triglyceride, lecithin, soap, sodium stearate, potassium stearate, ammonium stearate, sodium oleate, potassium oleate, ammonium oleate, sodium palmitate, potassium palmitate and ammonium palmitate.

According to some embodiments of the invention, the formulation in a form of a shampoo, the formulation further comprising at least one ingredient selected from the group consisting of a detergent or surfactant, a salt, an acid and a humectant.

According to some embodiments of the invention, the formulation further comprises an additional ingredient selected from the group consisting of a fragrant, a colorant, a polydimethylsiloxane, a quaternary ammonium derivative of natural guar gum, a protein, an amino acid, a vitamin, a provitamin, a botanical extract, a UV protectant, an antioxidant, a medically active agents and any combination thereof.

According to some embodiments of the invention, the formulation is in a form of a conditioner, the formulation further comprising at least one ingredient selected from the group consisting of a moisturizer/humectant, an emulsifier, a reconstructor, an acid and a buffer.

According to some embodiments of the invention, the formulation is further comprising an additional ingredient selected from the group consisting of a fragrant, a colorant, a hydrolyzed protein, a detangler, a thermal protector, a glossers, oil, an essential fatty acid, a cationic surfactant, a lubricant, a an fatty alcohol, a sequestrant, an antistatic agent, a preservative and any combination thereof.

According to some embodiments of the invention, the formulation is in a form of a hairspray, the further comprising at least one ingredient selected from the group consisting of a fixing agent, an alcohol, a surfactant, an emulsifier, a propellant, a polydimethylsiloxane, an alkylacrylamide/acrylate copolymer.

According to some embodiments of the invention, the formulation further comprises an additional ingredient selected from the group consisting of a fragrant, a colorant, a lubricant, an antistatic agent, hair setting polymer, a preservative and any combination thereof.

According to another aspect of some embodiments of the invention there is provided a process for preparing the hair care formulation as described herein, the process comprising: adding the liquid ovicidal/pediculicidal composition as described herein into a formulation of a hair care composition selected from the group consisting of a shampoo, a conditioner, a mask, a mousse, a gel, a hair spray, a lotion, a moisturizer, a tonic and a serum.

According to another aspect of some embodiments of the invention there is provided a method of eradicating a population of at least one ectoparasite in human hair, the method comprising: (a) applying a disinfesting effective amount of the hair care formulation of any of claims 1-22 to wet hair; (b) leaving the formulation on the hair and scalp for a pre-determined time period; and (c) rinsing the formulation from the hair.

According to some embodiments of the invention, the pre-determined time period ranges from 1 minute to 20 minutes.

According to some embodiments of the invention, the pre-determined time period ranges from 1 minute to 10 minutes.

According to some embodiments of the invention, the pre-determined time period ranges from 1 minute to 6 minutes.

According to some embodiments of the invention, the effective amount ranges from 10 grams to 30 grams of the hair care formulation.

According to some embodiments of the invention, the method is further comprising, subsequent to the applying, working the formulation through the hair and scalp.

According to another aspect of embodiments of the invention there is provided a liquid ovicidal/pediculicidal composition comprising: a film-forming carbohydrate, as described herein, as described herein, in an amount that ranges from about 5 weight percents to about 50 weight percents; and water, the composition being identified for eradicating a population of at least one ectoparasite, as described herein.

According to some embodiments of the invention, the liquid ovicidal/pediculicidal is further comprising oil, as described herein, in an amount that ranges from about 1 weight percent to about 20 weight percents of the composition.

According to some embodiments of the invention, the liquid ovicidal/pediculicidal is further comprising an emulsifier, as described herein, in an amount that ranges from about 0.5 weight percent to about 5 weight percents.

According to another aspect of embodiments of the invention there is provided a process for preparing the liquid/pediculicidal composition described herein, the process comprising: mixing the film-forming carbohydrate, the oil, if present, the emulsifier, if present, and the water.

According to another aspect of embodiments of the invention there is provided a use of the liquid/pediculicidal composition described herein in the manufacture of a hair care formulation for eradicating a population of at least one ectoparasite, as described herein

According to some embodiments of the invention, the eradicating a population of at least one ectoparasite is effected by: (a) applying a disinfesting effective amount of the hair care formulation to wet hair, as described herein; (b) leaving the formulation on the hair and scalp for a pre-determined time period, as described herein; and (c) rinsing the formulation from the hair.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein, the singular form “a”, “an” and “the” include plural references unless the context clearly dictates otherwise. For example, the term “a compound” or “at least one compound” may include a plurality of compounds, including mixtures thereof.

As used herein the term “about” refers to ±10.

The symbol “%”, as used herein, represents the phrase “percentage by weight” in general, and more specifically “percentage by weight of the total weight of a composition/mixture/formulation”, unless specified otherwise.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the terms “method” or “process” refer to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

### DESCRIPTION OF SPECIFIC EMBODIMENTS OF THE INVENTION

The present invention, in some embodiments thereof, relates to the field of cosmetics and household remedies for eradication of ectoparasites, and more particularly, but not exclusively, to anti-phthiraptera treatment compositions and methods using the same.

Before explaining at least one embodiment of the invention in detail, it is to be understood that the invention is not necessarily limited in its application to the details of construction and the arrangement of the components and/or methods set forth in the following description and/or illustrated in the Examples. The invention is capable of other embodiments or of being practiced or carried out in various ways. Also, it is to be understood that the phraseology and terminology employed herein is for the purpose of description and should not be regarded as limiting.

As discussed hereinabove, currently known lice-killing compositions are based on a pediculicidal which is typically a chemical compound that is toxic to the insect at concentrations which are relatively non-toxic to humans. The standards in clinical efficacy tests of a pediculicidal compound require a minimum of 85% death in the tested lice population, while effective compounds afford 92-95% kills among the lice. Mechanical removal by dense combs typically removes 35% of the population mainly due to the strong grips of the individual nymphs and adults and the limited accessibility of the comb to all parts of the infected areas. However, any less than 100% in eradicating an ectoparasite is almost futile as the pest population will recover in a matter of 1-2 weeks owing to the surviving part of the population and the unaffected ova. Furthermore, over the course of repetitive use the surviving lice present a far tougher challenge since they are more likely to develop resistance to the pediculicidal compound to some extent, a trait that would be amplified over the next generations at an exponential rate.

Therefore, while conceiving the present invention, the inventor has aimed at a solution which would afford multiple objectives, including pediculicidal and ovicidal activity as well as a pest behavior-altering effect, without exerting a toxic load on the treated subject. The rudimentary approach underlying the present invention was set to achieve a long-term pediculicidal and ovicidal activities, while affecting the behavior of the pest so as to make the common mechanical removal of the surviving population far more effective.

While reducing the present invention to practice, the inventor has designed and successfully practiced a non-toxic and benign film-forming composition that can exert a pediculicidal effect per-se on ova, nymph and adult individuals, and in parallel forces the surviving adult individuals into a more expose position, thereby sensitizing the survivors to standard mechanical combing. This combined multiple-faceted pediculicidal composition afforded a highly efficacious anti-ectoparasite activity.

Mere water encapsulation is known not to kill lice for at least 6 hours, and further not to loosen its grip on the skin and hair strands. The main effect of the film-forming agent (carbohydrate) practiced by the present inventor in combination with other ingredients in the liquid ovicidal/pediculicidal composition presented herein, is also to loosen the grip of the nymph and adult ectoparasite, and encapsulate the individuals, leading to death, presumably by suffocation. The grip-loosening effect renders the nymphs and adult ectoparasite more susceptible to mechanical removal by rinsing and by means of a dense comb. Therefore, the film-forming agent is selected such that it is completely benign to the treated subject, and can form a film on the treated area (for example, hair and scalp) at normal living conditions even when the treated area is partially dried or substantially dried. The resulting encapsulating coat formed by the composition is selected so as not to form a crystalline or powdery form at any stage of the treatment which may extend several hours at normal ambient conditions.

While further reducing the present invention to practice, the inventor has found that many edible or otherwise benign carbohydrates form a film when a solution thereof is dried, as opposed to forming powdery or crystalline sediments. Further research into an optimal composition has revealed that combining a film-forming carbohydrate with edible or otherwise benign oil in a concentrated aqueous solution affords the desired ovicidal/pediculicidal result, while being completely safe and even edible.

In order for the present ovicidal/pediculicidal composition to be effective and still agreeable for the user, it was recognized that a composition containing a film-forming carbohydrate should also be washable from the user's hair, namely be easy to rinse-off without leaving residues and other remnants thereof, which was and still is one of the major drawbacks of the old and well-known home-made and folkloric anti-lice remedies. This capacity to wash-off can be defined by a time period of less than 5 minutes, and even less than 1 minute, in which the composition will be rinsed-off from typical human hair of less than 60 cm in length, using a typical showerhead or another water delivery mechanism, flowing water at a rate of at least 5 liters per minute and at a typical showering temperature of less than 40° C.

It was further conceived that the ovicidal/pediculicidal composition can be used as a part of a hair care formulation, thereby rendering its use more agreeable and comfortable for the user, by being washable and having the more familiar form of an everyday use product.

While further reducing the invention to practice, washable and highly efficacious anti-ectoparasitic formulations were realized in the form of a hair care product, such as shampoos, hair conditioners, sprays and the likes.

In order to test this novel line of formulations, the inventor has used an exemplary shampoo formulation having a content of 10 weight percents glucose syrup and 1 percent canola oil, and the surprising results indicated that the heads of about 30 lice-infested children no longer had any remnants of lice after one or two uses of the shampoo.

Thus, according to one aspect of embodiments of the present invention, there is provided a novel hair care formulation.

According to some embodiments, the hair care formulation presented herein has a dual effect, one as an anti-ectoparasitic formulation, and one as a hair care formulation for everyday use.

The phrase “hair care formulation”, as use herein, refers to a formulation of substances which are combined so as to impart a beneficial or desired effect in hair, and include formulations known by their common names, such as a shampoo, a conditioner, a mask, a mousse, a gel, a hairspray, a lotion, a moisturizer, a tonic and a serum. Herein and throughout the term “hair care formulation” is also referred to, interchangeably as “formulation”.

According to embodiments of the present invention, the hair care formulation is an anti-ectoparasitic formulation, being capable of, and identified for use in, eradicating a population of ectoparasites.

According to some embodiments, the formulation is washable, as discussed hereinafter.

According to some embodiments, the hair care formulation described herein comprises from about 10 weight percents to about 60 weight percents of a liquid ovicidal/pediculicidal composition.

In some embodiments, the hair care formulation comprises from about 20 weight percents to about 60 weight percents of a liquid ovicidal/pediculicidal composition.

In some embodiments, a lower or higher concentration of the liquid composition is used, as long as the desired physicochemical properties, as detailed hereinbelow, of the formulation can be achieved.

In some embodiments, the liquid ovicidal/pediculicidal composition comprises:

a film-forming carbohydrate in an amount that ranges from about 5 weight percents to about 50 weight percents; and water.

Water is added in an amount required to complete said composition to 100 weight percents.

In some embodiments, the hair care formulation is washable within a time period of less than 5 minutes from hair at a length of 5 cm in a water shower stream of 20 liters per minute and a water temperature of 37° C.

In some embodiments, the hair care formulation is washable within a time period of less than 4 minutes, less than 3 minutes, less than 2 minutes, less than 1 minutes and even less than 30 seconds or less than 20 seconds, under the above-indicated conditions.

It is to be understood that while the hair care formulation described herein may require longer time periods for being washed off hair at higher or shorter lengths, the washability of the formulation is determined by the above-indicated conditions.

It is further to be understood that the phrase “water shower stream” describes stream of water coming out from all types of available shower heads, including fixed, positionable and handheld shower heads, shower heads equipped with a selector for multiple spray patterns, as long as the indicated conditions of water stream and temperature are maintained for determining the time period required for washing the formulation from the hair.

It is further to be understood that by “washable” it is meant that at least most, if not all, of a formulation that has been applied to the hair has been washed off the hair, such that the hair no longer feels “soapy” or otherwise wetted by ingredients other than water.

Commercially available hair care formulations for washing hair are typically washable at a time period of less than 1 minute, when determined at the above-indicated conditions.

In some embodiments, the hair care formulation described herein has similar washability as that of commercially available shampoos, conditioners and the like.

According to some embodiments, the liquid ovicidal/pediculicidal composition further comprises oil. In some embodiments, the composition comprises oil in an amount that ranges from about 1 weigh percents to about 20 weigh percents of the total weight of the composition.

Without being bound to any particular theory, it is assumed that coating of the insect body by the film-forming carbohydrate, blocks the air channels in the insect shell in all its life-cycle forms, thereby causing suffocation and death. This effective coating of the insect shell in all its life-cycle forms by an aqueous solution of a film-forming carbohydrate is believed to be so effective since the body of the insect comprises a polysaccharide (chitin), and the similar chemistry of the film-forming carbohydrate and the chitin allows the film-forming carbohydrate in the formulations and compositions presented herein to “wet” the insect nymph, adult and ova, and thereby kill it by suffocation.

As used herein, the phrase “liquid ovicidal/pediculicidal composition” describes a composition which is in a liquid state before use, and which when comes in contact with a nymph, adult or ova of an insect ectoparasite, such as phthiraptera, is capable of eradicating and/or devitalizing by exposure thereto and contact therewith a major portion of the population of the nymph, adult and/or ova thereof.

The liquid ovicidal/pediculicidal composition is also referred to herein throughout simply as “composition” or “liquid composition”.

By “eradicating and/or devitalizing” it is meant that the population of the nymph, adult and/or ova of the ectoparasite is reduced or eliminated to the extent that it can no longer be detected and can no longer re-appear without an external source of individuals which were not members of the eradicated population at the time of exposure to the ovicidal/pediculicidal composition according to some embodiments of the invention.

In some embodiments, eradicating a major portion of the ectoparasite's population is manifested by killing or devitalizing at least 60%, at least 70%, at least 80%, at least 90% and even 100% of the ectoparasites.

In some embodiments, the liquid ovicidal/pediculicidal composition comprises ingredients which are officially accepted as safe, or in other words, are classified as GRAS (Generally Accepted As Safe) by the national agencies which are responsible for regulations in the food and drug fields.

In some embodiments, the liquid ovicidal/pediculicidal composition described herein consists essentially of ingredients which are officially accepted as safe, as defined herein.

The film-forming carbohydrate included in the composition described herein as the active ingredient can be in an amount of, for example, 5, 10, 15, 20, 25, 30, 35, 40, 45 and 50 weight percents of the total weight of the composition.

The phrase “film-forming carbohydrate”, as used in the context of embodiments of the invention, refers to soluble carbohydrates or saccharides, which form a film on a surface when a solution thereof is dehydrated, dried, exposed to body temperature or to a combination of these conditions, as opposed to carbohydrates or saccharides which leave a powdery or crystalline residue or sediment when a solution thereof is exposed to any one or a combination of these conditions.

The term “carbohydrate” is used herein and in the art to describe a chemical substance, either naturally occurring or synthetic, which is mainly composed of carbon, hydrogen and oxygen atoms. The term “carbohydrate” is often used in the art to collectively describe saccharides and sugars, including monosaccharides, disaccharides, oligosaccharides, polysaccharides, and derivatives thereof, as detailed herein.

The term “monosaccharide”, as used herein and is well known in the art, refers to a simple form of a sugar that consists of a single saccharide unit which cannot be further decomposed to smaller saccharide building blocks or moieties. Most common examples of monosaccharides include glucose (dextrose), fructose, galactose, mannose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms.

Alternatively, the monosaccharide can be a monosaccharide derivative, in which the saccharide unit comprises one or more substituents other than hydroxyls. Such derivatives can be, but are not limited to, ethers, esters, acids, phosphates and amino saccharides.

Oligosaccharides are commonly defined in the art and herein as being composed of up to nine saccharide units (see, for example, Roberts, J. D., and Caserio, M. C., Basic Principles of Organic Chemistry (1964) p. 615). Representative examples include, without limitation, disaccharides such as, but not limited to, sucrose, maltose, lactose, and cellobiose; trisaccharides such as, but not limited to, mannotriose, raffinose and melezitose; and tetrasaccharides, such amylopectin, Syalyl Lewis X (SiaLex) and the like.

The term “polysaccharide” as used herein is meant to include compounds composed of 10 saccharide units and up of hundreds and even thousands of monosaccharide units per molecule, which are held together by glycoside bonds and range in their molecular weights from around 5,000 and up to millions of Daltons. Examples of common polysaccharides include, but are not limited to starch, glycogen, cellulose, gum arabic, agar and chitin.

The terms “carbohydrates”, “sugars” and “saccharides” are used interchangeably throughout the present disclosure unless specified otherwise.

Some sugars and saccharides are less likely to form powdery or crystalline residue or sediment from a solution thereof even when the solution is prepared from the purified, due to their multi-conformational structure. Sugars are also highly hygroscopic, making a dry and powdered form thereof hard to maintain at normal ambient conditions.

Most commercially available sugars and saccharides, and particularly those for everyday home use, are not purified so as to be regarded as a single chemical species, but tend to contain deliberate or consequential impurities as a side-product of their mass production. Other sugars and saccharides are available only as mixtures of several and sometime a great number of carbohydrate species, and some are known and sold as syrups.

Hence, a suitable “film-forming carbohydrate”, according to embodiments of the present invention, is selected such that when an aqueous solution thereof is exposed to dehydration and/or body temperature, a sticky film is formed.

Without being bound to any particular theory, it is suggested that film-forming carbohydrate as described herein form a sticky film when in contact with a body surface either due to its impure state (being a mixture of species at a certain species distribution), due to its hygroscopic character, or both.

According to some embodiments of the present invention, the film-forming carbohydrate may be composed of monosaccharides, disaccharides, oligosaccharides, and polysaccharides, as defined herein, each being pure, purified or impure; of crude saccharides; mixtures of saccharides; molasses; syrups; treacle and any combination thereof, as long as they are capable of forming a film when an aqueous solution thereof is exposed to dehydration and/or body temperature.

It is noted herein that some polysaccharides are known to form powdery and dry sediments rather than a sticky film even when present in non-uniform mixtures. For example, chitosan and some other by-products of chitin and chitin breakdown products are less suitable as film-forming carbohydrates in the context of the present embodiments.

Exemplary film-forming carbohydrates that are suitable for use in the context of embodiments of the invention include, but are not limited to, allose, altrose, arabinose, a beet-sugar syrup, a cane-sugar syrup, a corn syrup, cellobiose, deoxyribose, dextrose, erythrose, erythrulose, fructose, fructose syrup, fucose, fructan, galactitol, galactosamine, galactose, gentiobiose, gluconic acid, glucosamine, glucose, glucose syrup, glucuronic acid, glycerol (glycerine), gulose, a high fructose starch-based syrup (HFSS), a high-fructose corn syrup (HFCS), idose, inositol, isomalt, isoglucose, isomaltose, isomaltulose/palatinose, lactitol, lactose, lactulose, levulose, lyxose, maltitol, maltose, maltodextrin, maltotriose, mannitol, mannosamine, mannose, melibiose, nystose, propylene glycol, psicose, raffinose, rhamnose, ribose, ribulose, saccharose, sorbitol, sorbose, sucrose, tagatose, talose, threose, trehalose, turanose, xylitol, xylobiose, xylose, xylulose, α,β-trehalose (neotrehalose), β,β-trehalose (isotrehalose), β-allopyranose, and any mixtures, concentrates and syrups thereof.

Some of the aforementioned carbohydrates are presented in Table 1 below, stating their Chemical Abstracts Service registry number (CAS No.), chemical formula and molecular weight.

TABLE 1

| Name | CAS No. | Formula | MW |
|---|---|---|---|
| propylene glycol | 57-55-6 | C₃H₈O₂ | 76.09 |
| glycerol (glycerine) | 56-81-5 | C₃H₅(OH)₃ | 92.094 |
| erythrose | 583-50-6 | C₄H₈O₄ | 120.10 |
| threose | 95-43-2 | C₄H₈O₄ | 120.10 |
| erythrulose | 496-55-9 | C₄H₈O₄ | 120.104 |
| deoxyribose | 533-67-5 | C₅H₁₀O₄ | 134.13 |
| arabinose | 5328-37-0 | C₅H₁₀O₅ | 150.13 |
| lyxose | 1949-78-6 | C₅H₁₀O₅ | 150.13 |
| ribose | 200-059-4 | C₅H₁₀O₅ | 150.13 |
| ribulose | 488-84-6 | C₅H₁₀O₅ | 150.13 |
| xylose | L: 609-06-3 | C₅H₁₀O₅ | 150.13 |
| | D: 58-86-6 | | |
| | DL: 41247-05-6 | | |
| xylulose | 527-50-4 | C₅H₁₀O₅ | 150.13 |
| xylitol | 87-99-0 | C₅H₁₂O₅ | 152.15 |
| fucose | 2438-80-4 | C₆H₁₂O₅ | 164.16 |
| rhamnose | 10485-94-6 | C₆H₁₂O₅ | 164.16 |
| glucosamine | 3416-24-8 | C₆H₁₃NO₅ | 179.17 |
| mannosamine | 2636-92-2 | C₆H₁₃NO₅ | 179.17 |
| galactosamine | 7535-00-4 | C₆H₁₃NO₅ | 179.171 |
| galactose | 26566-61-0 | C₆H₁₂O₆ | 180.156 |
| glucose | L: 921-60-8 | C₆H₁₂O₆ | 180.156 |
| | D: 50-99-7 | | |
| mannose | 31103-86-3 | C₆H₁₂O₆ | 180.156 |
| psicose | 23140-52-5 | C₆H₁₂O₆ | 180.156 |
| allose | D: 2595-97-3 | C₆H₁₂O₆ | 180.16 |
| (beta-allopyranose) | L: 7635-11-2 | | |
| altrose | L:1949-88-8 | C₆H₁₂O₆ | 180.16 |
| | D:1990-29-0 | | |
| fructose | 57-48-7 | C₆H₁₂O₆ | 180.16 |
| gulose | L:6027-89-0 | C₆H₁₂O₆ | 180.16 |
| | D:4205-23-6 | | |
| idose | 2152-76-3 | C₆H₁₂O₆ | 180.16 |
| inositol | 87-89-8 | C₆H₁₂O₆ | 180.16 |
| sorbose | 87-79-6 | C₆H₁₂O₆ | 180.16 |
| tagatose | 17598-81-1 | C₆H₁₂O₆ | 180.16 |
| talose | 2595-98-4 | C₆H₁₂O₆ | 180.16 |
| galactitol | 608-66-2 | C₆H₁₄O₆ | 182.17 |
| sorbitol | 50-70-4 | C₆H₁₄O₆ | 182.17 |
| mannitol | 69-65-8 | C₆H₁₄O₆ | 182.172 |
| glucuronic acid | 6556-12-3 | C₆H₁₀O₇ | 194.14 |
| gluconic acid | 526-95-4 | C₆H₁₂O₇ | 196 |
| xylobiose | 6860-47-5 | C₁₀H₁₈O₉ | 282.24 |
| cellobiose | 16462-44-5 | C₁₂H₂₂O₁₁ | 342.30 |
| gentiobiose | 554-91-6 | C₁₂H₂₂O₁₁ | 342.30 |
| isomaltose | 499-40-1 | C₁₂H₂₂O₁₁ | 342.3 |
| isomaltulose/palatinose | 13718-94-0 | C₁₂H₂₂O₁₁ | 342.3 |
| β,β-trehalose (isotrehalose) | 499-23-0 | C₁₂H₂₂O₁₁ | 342.3 |
| lactose | 63-42-3 | C₁₂H₂₂O₁₁ | 342.3 |
| lactulose | 4618-18-2 | C₁₂H₂₂O₁₁ | 342.3 |
| maltose | 69-79-4 | C₁₂H₂₂O₁₁ | 342.3 |
| melibiose | 5340-95-4 | C₁₂H₂₂O₁₁ | 342.3 |
| α,(β-trehalose | 585-91-1 | C₁₂H₂₂O₁₁ | 342.3 |
| (neotrehalose) | | | |
| saccharose | 57-50-1 | C₁₂H₂₂O₁₁ | 342.3 |
| sucrose | 57-50-1 | C₁₂H₂₂O₁₁ | 342.3 |
| trehalose | 99-20-7 | C₁₂H₂₂O₁₁ | 342.3 |
| turanose | 547-25-1 | C₁₂H₂₂O₁₁ | 342.3 |
| isomalt | 64519-82-0 | C₁₂H₂₄O₁₁ | 344.31 |
| lactitol | 585-86-4 | C₁₂H₂₄O₁₁ | 344.31 |
| maltitol | 585-88-6 | C₁₂H₂₄O₁₁ | 344.31 |
| raffinose | 512-69-6 | C₁₈H₃₂O₁₆ | 504.42 |
| maltotriose | 1109-28-0 | C₁₈H₃₂O₁₆ | 504.44 |
| nystose | 13133-07-8 | C₂₄H₄₂O₂₁ | 666.58 |

While reducing the present invention to practice, as presented in the Examples section that follows, the inventor has found that the use of glucose syrup affords a highly efficacious liquid ovicidal/pediculicidal composition which can form a part of an effective anti-ectoparasitic hair care formulation.

The term “syrup” as used herein, refers to a thick and viscid and oftentimes saturated aqueous solution of a saccharide or a mixture or saccharides and other carbohydrates.

Glucose is formed in plants from carbon dioxide absorbed from the air using sun light as energy source. In the plant, part of the glucose is polymerized into long chains of glucose and stored as starch in granules as a reserve. In spring season, under stress and/or other conditions and stages in the life cycle of the plant, starch is broken down again by enzymes (e.g., amylase) to support new growth. This break down of starch can be imitated in factories by applying acid or enzymes to cooked aqueous solutions of starch. These reactions cause the starch to hydrolyze into a variety of mixtures of glucose and intermediates, and the common way to characterize these various mixtures is by their DE number. A DE number refers to the “Dextrose Equivalent” of a mixture, and is obtained by an analytical procedure which measures the reducing end-groups of the species in the mixture, thereby assigning a DE of 100 to pure glucose (glucose=dextrose) and a DE of 0 to pure starch.

Only glucose solution of high DE can crystallize easily and yield a powdery or granular form of a residue or sediment. The most commonly used and known crystallized product of glucose is dextrose monohydrate, with applications in, for example, medicine and as chewing tablets for athletes, therefore, for all intents and purposes of everyday life, dextrose monohydrate is pure glucose. A less purified product known as “Total Sugar” is produced by instant crystallizing a 97 DE syrup leaving no hydrol (mother liquor) to dispose off.

Lowering the DE, the syrup loose gradually its tendency to crystallize and below approximately 45 DE the syrup can be evaporated into a stable, non-crystallizing and auto sterile liquid due to a water activity level which is low so as to prevent any form of microbial life therein.

One of the most commercially abundant forms of glucose is therefore glucose syrup, which is prepared by having the starch hydrolyzed by acid or enzymes to 40-42 DE, and evaporated to a viscous liquid with a dry matter of 80-84 weight percents. Such a commodity is known as Glucose Syrup 80/42, or simply “42 DE”, owing to the 80 weight percents dry matter and DE value of 42. This standard product has a bland sweet taste; stores and ship well in drums or tank lorries. The relative sweetness of 42 DE to sucrose is 40-45 weight percents, and it find applications in canned fruit preserves, ice cream, bakery products, jam, soft drinks, candy and all kinds of confectionery, as well as a booster in the fermentation of alcohol.

Hence, in some embodiments, the film-forming carbohydrate is glucose syrup, such as, for example, Glucose Syrup 80/42.

In some embodiments, syrup of other carbohydrates is used.

The composition described herein comprises a film-forming carbohydrate in an aqueous solution, as described herein.

As further described hereinabove, in some embodiments, the composition further comprises oil.

Without being bound to any particular theory, it is postulated that the presence of oil in the composition contributes to the pediculicidal effect of the composition. It is postulated that together with the film-forming carbohydrate in water, oil forms a bi-physic system that can readily encapsulate the ectoparasite and cause loosening of its grip. In addition, it is suggested that the addition of oil attributes to the physicochemical properties of the obtained hair care formulations, as it may render the formulation washable, and may further be used to stabilize the formulation and/or composition described herein.

According to some embodiments, the liquid ovicidal/pediculicidal composition further includes oil in an amount that ranges from about 1 weight percents to about 20 weight percents of the composition. The oil can amount to about 1, 2, 6, 8, 10, 12, 14, 16, 18 and 20 weight percents.

Higher and lower concentrations of oil in the composition are also contemplated, as long as the desired physicochemical properties of the formulation are achieved.

It is well known that oil and aqueous (or other polar) solutions typically do not form a stable homogeneous solution when mixed. Without being bound to any particular theory, it is believed that the natural phenomenon of the organic phase (oil droplets) rising up from within the aqueous phase (an aqueous solution of a film-forming carbohydrate, such as for example, a solution of glucose in water), causes the film-forming carbohydrate solution to move toward the bodies and ova of the insect, causing the film-forming carbohydrate solution to coat the bodies and ova of the insects due to the similar chemistry of the film-forming carbohydrate and the chitinous shells of the insect nymph, adult and ova, as discussed hereinabove.

The ovicidal/pediculicidal compositions and hair care formulations described herein are formulated such that the individual parasitic specimen sinks or partly sinks in the liquid composition or hair care formulation due to the unique emulsion or otherwise bi-phasic combination of liquids having favorable characteristics of surface tension (wettability) and specific gravity with respect to the insect shell and body mass and density. Without being bound to any particular theory, it is assumed that the pediculicidal and ovicidal activities are effected, at least in part, by encapsulating the ectoparasite in a film, leading to loss or weakening of its grip and eventually leading to death by suffocation.

The oil can be any benign and possibly edible oil, so as not to involve toxic elements in the composition; however the oil is not required to be edible as long as it is safe for external use in mammalian subjects under indicated conditions.

Some aromatic oils and other essential oils are known to have some anti-insect activity, insect repellent activity and even insecticidal activity. In the context of some embodiments of the invention, such oils having anti-ectoparasitosis activity are highly suitable for use in the compositions presented herein. Still, it is noted that for commercial reasons and other industrial and marketing considerations, some aromatic and essential oils may be selected for the present composition in small amounts while the bulk of the oil may be selected from more commercially and/or economically available oils since the oil in the present composition can merely be used to form a bi-phasic system with the aqueous solution of the film-forming carbohydrate.

The oil can be a single type of oil or a combination of more than one type of oil. Exemplary oils that are suitable for use in the context of embodiments of the invention include, but are not limited to, acai oil, agarwood oil, algae oil, allspice oil, almond oil, amaranth oil, amur cork tree fruit oil, anise oil, apple seed oil, apricot oil, argan oil, artichoke oil, avocado oil, babassu oil, balanos oil, basil oil, bay leaf oil, ben (moring a oleifera) oil, bergamot oil, blackcurrant seed oil, bladderpod oil, borage seed oil, borneo tallow nut oil, bottle gourd oil, brucea javanica oil, buffalo gourd oil, burdock (bur) oil, camphor oil, candlenut (kukui nut) oil, cannabis oil, canola oil, cape chestnut (yangu) oil, carob pod oil, carrot seed oil, cashew oil, cassia oil, castor oil, cedar oil, celery oil, chamomile oil, chaulmoogra oil, cinnamon oil, clary sage oil, clove oil, cocklebur oil, cocoa oil, coconut oil, coconut oil, cohune oil, copaiba oil, copra oil, coriander seed oil, corn oil, cottonseed oil, crambe oil, cumin oil, cuphea oil, dammar oil, dika oil, eucalyptus oil, evening primrose oil, false flax (camelina sativa) oil, flax seed oil, frankincense oil, galangal oil, ginger oil, grape seed oil, grapefruit oil, hazelnut oil, hemp oil, honge oil, hops oil, hyssop oil, jasmine oil, jatropha oil, jojoba oil, juniper oil, kapok seed oil, lallemantia oil, lavender oil, lemon grass oil, lemon oil, lime oil, linseed oil, macadamia oil, mango oil, manuka oil, marjoram oil, manila oil, meadowfoam seed oil, melaleuca oil, mongongo nut oil, mowrah oil, mustard oil, mustard oil, myrrh oil, neem oil, nutmeg oil, nutmeg oil, ojon oil, okra seed oil, olive oil, orange oil, oregano oil, palm oil, papaya seed oil, patchouli oil, peanut oil, pecan oil, peppermint oil, pequi oil, perilla seed oil, pine nut oil, pine oil, pistachio oil, poppy seed oil, prune kernel oil, pumpkin seed oil, quinoa oil, radish oil, ramtil oil, rapeseed oil, rice bran oil, rose hip seed oil, rose oil, rosemary oil, rosewood oil, royle oil, sacha inchi oil, safflower oil, sage oil, salicornia oil, sandalwood oil, sassafras oil, scented geranium oil, sea buckthorn oil, sesame oil, shea oil, snowball seed oil, soybean oil, spearmint oil, stillingia oil, sunflower oil, tall oil, tamanu oil, tangerine oil, tea seed oil, tea tree oil, thistle oil, thyme oil, tomato seed oil, tocopherol (vitamin E), tocopheryl acetate, tonka bean (cumaru) oil, turmeric oil, tung oil, valerian oil, vernonia oil, walnut oil, watermelon seed oil, wheat germ oil, wintergreen oil and ylang-ylang oil.

In some embodiments, the oil is grape seed oil.

In some embodiments, the oil is canola oil and/or jojoba oil.

In some embodiments, the oil further comprises rosemary oil, turmeric oil, and any other oil that exhibits an anti-ectoparasitosis activity.

The choice of oil can be made according to the taste and preference of the user, and according to the mode of use of the composition. For example, the composition can be incorporated into a hair care product, such as a shampoo having a fragrance therein, the choice of oil should take into account the desired final smell of the product, and possibly be an odorless oil. On the same lines, an odorless shampoo composition may relay of the selection of fragrant oil for its smell, hence essential aromatic oil should be selected.

According to embodiments of the present invention, the liquid ovicidal/pediculicidal composition further includes an emulsifier.

The term “emulsifier” (also known as a surfactant or a surface active agent), as used herein, refers to a substance which stabilizes an emulsion.

The emulsion, as commonly known, is provided by preparing and mixing two solutions, one being the aqueous phase (water-based phase) and another being the organic phase (oil-based phase), so as to disperse one phase in the other.

According to some embodiments, an emulsion is an “oil-in-water” emulsion, wherein droplets of the oil are dispersed in the continuous aqueous phase.

In some embodiments, the amount of the emulsifier ranges from about 0.5 weight percents to about 5 weight percents of the total weight of the composition.

Exemplary emulsifiers that are suitable for use in the context of embodiments of the invention include, but are not limited to, sodium dodecyl sulfate, a phospholipid, a glycolipid, a triglyceride, lecithin, soap, sodium stearate, potassium stearate, ammonium stearate, sodium oleate, potassium oleate, ammonium oleate, sodium palmitate, potassium palmitate and ammonium palmitate.

In some embodiments, the liquid ovicidal/pediculicidal composition presented herein is packaged in a packaging material and is identified in print, in or on the packaging material, for use in a treatment against insect ectoparasites in general and human head lice in particular.

According to an aspect of some embodiments of the invention there is provided a process of preparing the liquid ovicidal/pediculicidal composition described herein, which is effected by adding a pre-determined amount of the film-forming carbohydrate to water, optionally adding a predetermined amount of the oil, optionally adding a predetermined amount of the emulsifier, adding water to the final weight of the composition, and mixing the resulting mixture so as to obtain a homogeneous mixture thereof.

In some embodiments, the liquid ovicidal/pediculicidal composition presented herein is used in the manufacture of a hair care formulation which is identified for use against insect ectoparasites in general and human head lice in particular, as described herein.

When incorporating the liquid ovicidal/pediculicidal composition presented herein into a hair care product (a hair care formulation), the obtained hair care formulation is regarded as an ovicidal/pediculicidal hair care formulation with the liquid ovicidal/pediculicidal composition presented herein as an active ingredient. In some embodiments, the amount of the liquid ovicidal/pediculicidal composition which is incorporated as a single liquid blend of ovicidal/pediculicidal composition described hereinabove, ranges from about 10 weight percents to about 60 weight percents of the total weight of the hair care formulation, as detailed hereinabove.

A hair care formulation can be formulated according to well-established experience accumulated in the art, into a variety of products, such as shampoos, conditioners, masks, mousses, gels, sprays, lotions, moisturizers, tonics serums and the likes. Hence, according to some embodiments of the present invention, the hair care formulation which is infused with the anti-ectoparasite composition presented herein, is a shampoo product for the eradication of, for example, head lice in humans.

As is well known in the art, a shampoo is a hair care product which is typically used for the removal of bodily secreted fats and oils, dead skin particles, dandruff and other bodily debris, environmental dirt and other pollutants and other contaminant particles that gradually build up in the hair and on the scalp. The general objective of shampoos is to remove the unwanted build-up without stripping off the hair which may damage it or harm the scalp skin.

Hence, a shampoo, when lathered with water, is a surfactant, which, while cleaning the hair and scalp, can remove the natural oils (sebum) which lubricate the hair shaft. Typical shampoo ingredients include one or more, and any combination of:

A detergent, which is typically a salt of a fatty acid, such as an ammonium or sodium form of lauryl or laureth sulfate;

A salt such as ammonium chloride;

Acids, such as alpha-hydroxy acids;

Humectants such as glycols;

Polymeric organosilicon and polydimethylsiloxane, such as dimethicone;

Quaternary ammonium derivatives of natural guar gum, such as Guar hydroxypropyltrimonium chloride;

Proteins and/or amino acids;

Vitamins and provitamins;

Botanical extracts;

UV protectant;

Antioxidants; and

Optionally, additional medications or other medically active agents (anti-dandruff etc.).

According to some embodiments of the present invention, the hair care formulation which is infused with the anti-ectoparasite composition presented herein, is a hair conditioning product, or a hair conditioner for the eradication of, for example, head lice.

As well known in the art, a hair conditioner, referred to herein and throughout as a conditioner, is a hair care product which is typically used for altering the texture and appearance of human hair. Typically, conditioners include one or more, and any combination of:

Moisturizers and humectants for maintaining a relatively high level of moisture in the hair;

Reconstructors in the form of hydrolyzed proteins, which are said to penetrate the hair and strengthen its structure through biopolymer crosslinking;

Acidifiers and buffers which maintain the conditioner's pH at about 2.5-3.5, which is said to cause scaly scalp surface to tighten-up due to strengthening of the hydrogen bonds between the keratin molecules.

Detanglers in the form of synthetic or natural polymers which modify the hair surface by coating it;

Thermal protectors, in the form of heat-absorbing polymers, shielding the hair against excessive heat, caused by, e.g., blow-drying or curling irons or hot rollers.

Glossers, which are light-reflecting chemicals that can bind to the hair surface, such as silicone-bases polymers such as dimethicone or cyclomethicone;

Oils and other essential fatty acids resembling sebum, which can help dry/porous scalp-skin and hair to become more soft and pliable. The scalp produces a natural oil called sebum;

Surfactants such as cationic surfactants, which don't wash off completely in water, because their hydrophilic ends strongly bind to keratin in the hair;

Lubricants, such as fatty alcohols, panthenol, dimethicone and the likes;

Sequestrants, for better function in hard water; Antistatic agents; and

Preservatives.

Other optional ingredients which are known to be used in hair care products, subject to particular requirements and preference, may be used in the hair care formulations presented herein, and include:

Alkalinity Source—Increases the alkalinity of the product to aid dissolution of dirt; Antidandruff Agent—Controls dandruff;

Antifoaming Agent—Suppresses the formation of excess foam during production and/or consumer use;

Antimicrobial Agent—Added to cosmetic products to help reduce the activities of micro-organisms on the skin or body;

Antioxidant—Inhibits the product reacting with oxygen, which can cause product spoilage;

Antiperspirant Agent—The active component that reduces perspiration;

Anti-redeposition Agent—Prevents dirt in the wash solution being re-deposited on the clothes;

Antistatic Agent—Prevents static charges on human surfaces e.g. skin/hair or on clothes;

Astringent—Induces a tightening or a tingling sensation on the skin;

Binder—Substances which provide adhesive properties so that the solids stick together e.g. tablets, eye shadow;

Botanicals—Ingredients derived from plants e.g. Aloe Vera;

Buffering Agent—Adjusts or stabilizes the acidity/alkalinity of a product;

Builder—Reduces the effect of water hardness by binding calcium and magnesium ions;

Bulking Agent—Increases the volume of a product through dilution, so that it can be applied at the correct concentration;

Colorant—Colors the skin/hair and/or the product;

Cooling Agent—Makes the skin feel cooler;

Corrosion Inhibitor—Prevents corrosion of the packaging;

Deodorant Agent—Reduces or masks unpleasant body odors;

Emulsifier—Used in small quantities that help to form or stabilize immiscible liquids in an emulsion;

Emulsion Stabilizer—Improves stability of an emulsion and increases shelf life of product;

Evaporation Aid—Helps the product dry faster after application;

Exfoliating Agent—Helps remove dead skin cells from the surface;

Film Former—Makes product form a uniform film when applied to a surface;

Fixing Agent—Maintains the hold/shape of a hair style;

Fragrance—Makes the product smell nice in the packaging and during use;

Hair Conditioning Agent—Enhances the appearance and texture of the hair, improves gloss, sheen and texture of damaged hair, and facilitates styling;

Hair Waving/Straightening Agent—Modifies hair fibers to facilitate configuration changes;

Humectant—Holds/retains water in the product, and/or increases the moisture content in the top layers of the skin;

Hydrotrope—Increases the solubility of the detergent in the product;

Opacifier—Reduces the transparency/translucency of a product, may give a pearly appearance;

Oxidizing Agent—Remove hair colorants or stabilize the hair after perming;

Pearlescer—Gives sheen to a liquid, which can be seen as flow lines when poured;

pH Adjuster—Adjusts the acidity/alkalinity of products;

Plasticizer—Reduces product brittleness and modifies flow properties;

Preservative—Protects products from microbial growth and spoilage from accidental contamination by the consumer during use;

Propellant—Delivers the product from an aerosol;

Reducing Agent—Changes the chemical nature of another substance by adding hydrogen or removing oxygen;

Sequestrant—Prevents free metal ions from causing any adverse effects on product performance, appearance, or stability by reacting with them;

Skin Conditioning Agent—Help to keep the skin soft, smooth, pliable and healthy looking

Skin Protectant—Protects the skin from the influences of the external environment;

Slip Modifier—Enhances the physical flow characteristics of the product;

Softness Extender—Improves the effectiveness of a softening agent;

Solubilizer—Improves the solubility of one or more product ingredients;

Solvent—Dissolves other ingredients/materials;

Spray Modifier—Modifies the spray characteristics of a spray;

Stabilizing Agent—Maintains ingredient or formulation stability;

Structurant—Gives form or shape to final products;

Sunscreen Agent—Filters out harmful UV rays;

Surface Modifiers—Change the physical characteristics of the surface;

Surfactant—Changes the surface tension of water to assist cleansing, wetting surfaces, foaming and emulsifying (the suspension of one liquid evenly within another);

Suspending/Dispersing Agent—Aids the even dispersal of a solid within a liquid;

UV Absorber—Filters UV rays to protect the product or skin/hair from UV damage;

Viscosity Controlling Agent—Controls the thickness of the product.

The hair care compositions of the present invention may be formulated into a wide variety of product types, including shampoos, conditioners, mousses, gels, lotions, tonics, sprays and the likes. The additional components required to formulate such products varies with product type and can be chosen by one skilled in the art of hair care formulation. The following is a description of some examples of these additional components.

Exemplary surfactants useful in the compositions and/or formulations of embodiments of the present invention include, but are not limited to, anionic, nonionic, cationic, zwitterionic and amphoteric surfactants.

Surfactants are preferred optional ingredients in the hair care formulations described herein, particularly shampoo and conditioner products. When present, the surfactant comprises from about 0.05 weight percents to about 50 weight percents of the total weight of the formulation. For a shampoo, the level is typically from about 10 to about 30 weight percents, or alternatively from about 12 to about 25 weight percents of the total weight of the formulation.

For conditioners the level of surfactant is typically from about 0.2 weight percents to about 3 weight percents.

Exemplary synthetic anionic detergents useful herein, particularly for the shampoo formulations, include, but are not limited to, alkyl and alkyl ether sulfates. These materials have the respective formulae ROSO₃M and RO(C₂H₄O)×SO₃M wherein R is alkyl or alkenyl of about 10 to about 20 carbon atoms, x is 1 to 10, and M is a water-soluble cation such as ammonium, sodium, potassium and triethanolamine. The alkyl ether sulfates useful in the context of the present embodiments are condensation products of ethylene oxide and monohydric alcohols having about 10 to about 20 carbon atoms. Typically, R has from 12 to 18 carbon atoms in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with to 10, and especially 3, molar proportions of ethylene oxide and the resulting mixture of molecular species, having, for example, an average of 3 moles of ethylene oxide per mole of alcohol, is sulfated and neutralized.

Some examples of alkyl ether sulfates according to embodiments of the present invention include, but are not limited to, sodium coconut alkyl triethylene glycol ether sulfate; lithium tallow alkyl triethylene glycol ether sulfate; and sodium tallow alkyl hexaoxyethylene sulfate. Extensively used alkyl ether sulfates are those comprising a mixture of individual compounds, said mixture having an average alkyl chain length of from about 12 to 16 carbon atoms and an average degree of ethoxylation of from about 1 to 4 moles of ethylene oxide. Such a mixture also comprises from about 0 to 20 weight percents C₁₂₋₁₃ compounds; from 60 to 100 weight percents of C₁₄₋₁₅₋₁₆ compounds, from about 0 to 20 weight percents of C₁₇₋₁₈₋₁₉ compounds; from about 3 to 30 weight percents of compounds having a degree of ethoxylation of 0; from about 45 to 90 weight percents of compounds having a degree of ethoxylation of from 1 to 4; from about 10 to 25 weight percents of compounds having a degree of ethoxylation of from 4 to 8; and from about 0.1 to 15 weight percents of compounds having a degree of ethoxylation greater than 8.

Another suitable class of anionic surfactants are the water-soluble salts of the organic, sulfuric acid reaction products of the general formula: R₁—SO₃-M, wherein R₁ is selected from the group consisting of a branched or unbranched hydrocarbon chain, saturated aliphatic hydrocarbon radical having from 8 to 24 carbon atoms, or 12 to 18 carbon atoms; and M is a cation. According to some embodiments, anionic surfactants are the salts of an organic sulfuric acid reaction product of a hydrocarbon of the methane series, including iso-, neo-, ineso-, and n-paraffins, having 8 to 24 carbon atoms, or 12 to 18 carbon atoms and a sulfonating agent e.g., SO₃, H₂SO₄, oleum, obtained according to known sulfonation methods, including bleaching and hydrolysis. Other examples include alkali metal and ammonium sulfonated C₁₂₋₁₈-n-paraffins.

Additional examples of anionic synthetic detergents according to other embodiments of the present invention include the reaction products of fatty acids esterified with isethionic acid and neutralized with sodium hydroxide where, for example, the fatty acids are derived from coconut oil; sodium or potassium salts of fatty acid amides of methyl tauride in which the fatty acids, for example, are derived from coconut oil. Other anionic synthetic detergents of this variety are set forth in, for example, U.S. Pat. Nos. 2,486,921, 2,486,922 and 2,396,278.

Still other anionic synthetic detergents include the class designated as succinamates. This class includes such surface active agents as disodium N-octadecylsulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Other suitable anionic detergents utilizable herein are olefin sulfonates having about 12 to about 24 carbon atoms. The term “olefin sulfonates” is used herein to mean compounds which can be produced by the sulfonation of α-olefins by means of uncomplexed sulfur trioxide, followed by neutralization of the acid reaction mixture in conditions such that any sulfones which have been formed in the reaction are hydrolyzed to give the corresponding hydroxy-alkanesulfonates. The sulfur trioxide can be liquid or gaseous, and is usually, but not necessarily, diluted by inert diluents, for example by liquid SO₂, chlorinated hydrocarbons, etc., when used in the liquid form, or by air, nitrogen, gaseous SO₂, etc., when used in the gaseous form.

The α-olefins from which the olefin sulfonates are derived are mono-olefins having 12 to 24 carbon atoms, or 14 to 16 carbon atoms. According to some embodiments, these are straight chain olefins, and include 1-olefins include 1-dodecene; 1-tetradecene; 1-hexadecene; 1-octadecene; 1-eicosene and 1-tetracosene. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process.

The α-olefins from which the olefin sulfonates are derived are mono-olefins having 12 to 24 carbon atoms or 14 to 16 carbon atoms. According to some embodiments, they are straight chain olefins which include 1-olefins include 1-dodecene; 1-tetradecene; 1-hexadecene; 1-octadecene; 1-eicosene and 1-tetracosene.

In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins and impurities in the olefin stock and side reactions during the sulfonation process.

A specific α-olefin sulfonate mixture of the above type is described more fully in the U.S. Pat. No. 3,332,880, the disclosure of which is incorporated herein by reference.

Another class of anionic organic detergents are the β-alkyloxy alkane sulfonates, as described in the art, e.g., U.S. Pat. No. 4,983,383, which is incorporated herein by reference. Non-limiting examples of β-alkyloxy-alkane-1-sulfonates, or alternatively 2-alkyloxy-alkane-1-sulfonates, having low hardness (calcium ion) sensitivity useful herein to provide superior cleaning levels under household hair washing conditions include: potassium-β-methoxydecanesulfonate, sodium 2-methoxy-tridecane sulfonate, potassium 2-ethoxytetradecylsulfonate, sodium 2-isopropoxyhexadecylsulfonate, lithium 2-t-butoxytetradecyl-sulfonate, sodium β-methoxyoctadecylsulfonate, and ammonium β-n-propoxydofonate.

Many additional non-soap synthetic anionic surfactants are described in McCutcheon's Detergents and Emulsifiers, 1984 Annual, published by Allured Publishing Corporation, as well as U.S. Pat. No. 3,929,678, which discloses many other anionic as well as other surfactant types, all of which are incorporated herein by reference.

Non-ionic surfactants, which are extensively used in combination with an anionic, amphoteric or zwitterionic surfactant, can be broadly defined as compounds produced by the condensation of alkylene oxide groups (hydrophilic in nature) with an organic hydrophobic compound, which may be aliphatic or alkyl aromatic in nature. Examples of some classes of non-ionic surfactants include

Non-ionic surfactants which are polyethylene oxide condensates of alkyl phenols, e.g., the condensation products of alkyl phenols having an alkyl group containing from about 6 to 12 carbon atoms in either a straight chain or branched chain configuration, with ethylene oxide, the ethylene oxide being present in amounts equal to 10 to 60 moles of ethylene oxide per mole of alkyl phenol. The alkyl substituent in such compounds may be derived from polymerized propylene, diisobutylene, octane, or nonane, for example.

Non-ionic surfactants derived from the condensation of ethylene oxide with the product resulting from the reaction of propylene oxide and ethylene diamine products which may be varied in composition depending upon the balance between the hydrophobic and hydrophilic elements which is desired. For example, compounds containing from about 40 to about 80 weight percents polyoxyethylene and having a molecular weight of from about 5,000 to about 11,000 resulting from the reaction of ethylene oxide groups with a hydrophobic base constituted of the reaction product of ethylene diamine and excess propylene oxide, said base having a molecular weight of the order of 2,500 to 3,000, are satisfactory.

Non-ionic surfactants which are condensation products of aliphatic alcohols having from 8 to 18 carbon atoms, in either straight chain or branched chain configuration, with ethylene oxide, e.g., a coconut alcohol ethylene oxide condensate having from 10 to 30 moles of ethylene oxide per mole of coconut alcohol, the coconut alcohol fraction having from 10 to 14 carbon atoms.

Non-ionic surfactants that are long chain tertiary amine oxides, which include, for example, dimethyl-dodecylamine oxide, oleyldi(2-hydroxyethyl) amine oxide, dimethyl-octylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldi-methylamine oxide, 3-dodecoxy-2-hydroxypropyldi(3-hydroxypropyl)amine oxide, dimethylhexadecylamine oxide.

Non-ionic surfactants that are long chain tertiary phosphine oxides, which include, for example, dodecyldimethylphosphine oxide, tetradecyldimethylphosphine oxide, tetradecylmethylethylphosphine oxide, 3,6,9,-trioxaoctadecyldimethylphosphine oxide, cetyldimethylphosphine oxide, 3-dodecoxy-2-hydroxypropyldi(2-hydroxyethyl) phosphine oxide, stearyldimethylphosphine oxide, cetylethylpropylphosphine oxide, oleyldiethylphosphine oxide, dodecyldiethylphosphine oxide, tetradecyldiethylphosphine oxide, dodecyldipropylphosphine oxide, dodecyldi(hydroxymethyl)phosphine oxide, dodecyldi(2-hydroxyethyl)phosphine oxide, tetradecylmethyl-2-hydroxypropylphosphine oxide, oleydimethylphosphine oxide, 2-hydroxydodecyldimethylphosphine oxide.

Non-ionic surfactants that are long chain dialkyl sulfoxides containing one short chain alkyl or hydroxy alkyl radical of to about 3 carbon atoms (usually methyl) and one long hydrophobic chain which contain alkyl, alkenyl, hydroxy alkyl, or keto alkyl radicals containing from about 8 to about 20 carbon atoms, from 0 to about 10 ethylene oxide moieties and from 0 to 1 glyceryl moiety. Some examples include octadecyl methyl sulfoxide, 2-ketotridecyl methyl sulfoxide, 3,6,9,-trixaoctadecyl 2-hydroxyethyl sulfoxide, dodecyl methyl sulfoxide, oleyl 3-hydroxypropyl sulfoxide, tetradecyl methyl sulfoxide, 3-methoxytridecyl methyl sulfoxide, 3-hydroxytridecyl methyl sulfoxide, 3-hydroxy-4-dodecoxybutyl methyl sulfoxide.

Cationic surfactants useful in the formulations of the present invention, particularly the conditioner compositions, contain amino or quaternary ammonium hydrophilic moieties which are positively charged when dissolved in the aqueous composition of the present invention. Cationic surfactant vehicle materials among those useful herein are disclosed in the following documents, all incorporated by reference herein: M.C. Publishing Co., McCutcheon's Detergents & Emulsifiers, (North American edition 1979); Schwartz, et al., Surface Active Agents, Their Chemistry and Technology, New York: Interscience Publishers, 1949; U.S. Pat. No. 3,155,591; U.S. Pat. No. 3,929,678; U.S. Pat. No. 3,959,461; and U.S. Pat. No. 4,387,090. When included in the formulations of the present invention, the cationic surfactant is present at from about 0.05 weight percents to about 5 weight percents.

Quaternary ammonium-containing cationic surfactant materials and salts thereof useful herein include dialkyldimethylammonium chlorides, wherein in the alkyl groups have from 12 to 22 carbon atoms and are derived from long-chain fatty acids, such as hydrogenated tallow fatty acid (Tallow fatty acids give rise to quaternary compounds wherein R 1 and R 2 have predominately from 16 to 18 carbon atoms), ditallowedimethyl ammonium chloride, ditallowedimethyl ammonium methyl sulfate, dihexadecyl dimethyl ammonium chloride, di(hydrogenated tallow) dimethyl ammonium chloride, dioctadecyl dimethyl ammonium chloride, dieicosyl dimethyl ammonium chloride, didocosyl dimethyl ammonium chloride, di(hydrogenated tallow) dimethyl ammonium acetate, dihexadecyl dimethyl ammonium chloride, dihexadecyl dimethyl ammonium acetate, ditallow dipropyl ammonium phosphate, ditallow dimethyl ammonium nitrate, di(coconutalkyl) dimethyl ammonium chloride, and stearyl dimethyl benzyl ammonium chloride. Ditallow dimethyl ammonium chloride, dicetyl dimethyl ammonium chloride, stearyl dimethyl benzyl ammonium chloride and cetyl trimethyl ammonium chloride are more useful in some embodiments of the present invention as quaternary ammonium salts, while in other embodiments it is di-(hydrogenated tallow) dimethyl ammonium chloride.

Salts of primary, secondary and tertiary fatty amines are also useful cationic surfactant vehicle materials in the context of the present invention. The alkyl groups of such amines preferably have from 12 to 22 carbon atoms, and may be substituted or unsubstituted. Secondary and tertiary amines are also useful, and more so tertiary amines. Such amines, useful herein, include stearamido propyl dimethyl amine, diethyl amino ethyl stearamide, dimethyl stearamine, dimethyl soyamine, soyamine, myristyl amine, tridecyl amine, ethyl stearylamine, N-tallowpropane diamine, ethoxylated stearylamine, dihydroxy ethyl stearylamine, and arachidylbehenylamine. Suitable amine salts include the halogen, acetate, phosphate, nitrate, citrate, lactate and alkyl sulfate salts. Such salts include stearylamine hydrochloride, soyamine chloride, stearylamine formate and N-tallowpropane diamine dichloride and stearamidopropyl dimethylamine citrate. Cationic amine surfactants included among those useful in the present invention are disclosed in U.S. Pat. No. 4,275,055, which is incorporated by reference herein.

Zwitterionic surfactants, useful in many hair care formulations according to embodiments of the present invention, such as shampoos and conditioners, can be exemplified by those which can be broadly described as derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate, or phosphonate. Examples of zwitterionic surfactants include, 4-(N,N-di(2-hydroxyethyl)-N-octadecylammonio)-butane-1-carboxylate; 5-(S-3-hydroxypropyl-S-hexadecylsulfonio)-3-hydroxypentane-1-sulfate; 3-(P,P-diethyl-P-3,6,9-trioxatetradexocylphosphonio)-2-hydroxypropane-1-phosphate; 3-(N,N-dipropyl-N-3-dodecoxy-2-hydroxypropylammonio)-propane-1phosphonate; 3-(N,N-dimethyl-N-hexadecylammonio)propane-1-sulfonate; 3-(N,N-dimethyl-N-hexadecylammonio)-2-hydroxypropane-1-sulfonate; 4-(N,N-di(2-hydroxyethyl)-N-(2-hydroxydodecyl)ammonio)-butane-1carboxylate; 3-(S-ethyl-S-(3-dodecoxy-2-hydroxypropyl)sulfonio)-propane-1-phosphate; 3-[P,P-dimethyl-P-dodecylphosphonio)-propane-1-phosphonate; and 5-(N,N-di(3-hydroxypropyl)-N-hexadecylammonio)-2-hydroxy-pentane-1-sulfate.

Other zwitterionics such as betaines are also useful in the present invention. Examples of betaines useful herein include the high alkyl betaines such as coco dimethyl carboxymethyl betaine, lauryl dimethyl carboxymethyl betaine, lauryl dimethyl alpha-carboxy-ethyl betaine, cetyl dimethyl carboxymethyl betaine, lauryl bis-(2-hydroxy-ethyl) carboxymethyl betaine, stearyl bis-(2-hydroxy-propyl) carboxymethyl betaine, oleyl dimethyl gamma-carboxypropyl betaine, lauryl bis-(2-hydroxypropyl)alphacarboxyethyl betaine, etc. The sulfobetaines may be represented by coco dimethyl sulfopropyl betaine, stearyl dimethyl sulfopropyl betaine, lauryl dimethyl sulfoethyl betaine, lauryl bis-(2-hydroxy-ethyl) sulfopropyl betaine and the like; amido betaines and amidosulfobetaines, wherein the RCONH(CH₂)₃ radical is attached to the nitrogen atom of the betaine are also useful in this invention. The amido betaines are more useful in some embodiments of the present invention.

Examples of amphoteric surfactants which can be used in the formulations according to embodiments of the present invention, are those which can be broadly described as derivatives of aliphatic secondary and tertiary amines in which the aliphatic radical can be straight chain or branched and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products sold under the trade name “Miranol” and described in U.S. Pat. No. 2,528,378.

The above-mentioned surfactants can be used either alone or in combination in the hair care formulations according to some embodiment of the present invention. In some embodiments, surfactants such as alkyl sulfates, ethoxylated alkyl sulfates and mixtures thereof are utilized.

Where the hair care formulations are conditioner formulations and more so hair gel formulations, some optional embodiments include gel vehicle materials. The vehicle comprises two essential components: a lipid vehicle material and a cationic surfactant vehicle material. Such gel-type vehicles are generally described in the following documents, all incorporated by reference herein: Barry, “The Self Bodying Action of the Mixed Emulsifier Sodium Dodecyl Sulfate/Cetyl Alcohol”, 28 J. of Colloid and Interface Science, 82-91 (1968); Barry, et al., “The Self-Bodying Action of Alkyltrimethylammonium Bromides/Cetostearyl Alcohol Mixed Emulsifiers; Influence of Quaternary Chain Length”, 35, J. of Colloid and Interface Science, 689-708 (1971); and Barry, et al., “Rheology of Systems Containing Cetomacrogol 1000 Cetostearyl Alcohol, I. Self Bodying Action”, 38 J. of Colloid and Interface Science, 616-625 (1972).

The vehicles may incorporate one or more lipid materials, referred to herein as comprising a “lipid vehicle material”, singly or in combination, which are essentially water-insoluble, and contain hydrophobic and hydrophilic moieties. Lipid vehicle materials include naturally or synthetically-derived acids, acid derivatives, alcohols, esters, ethers, ketones, and amides with carbon chains of from 12 to 22, or from 16 to 18, carbon atoms in length. Fatty alcohols and fatty esters are particularly useful, and more so fatty alcohols.

Lipid vehicle materials among those useful herein are disclosed in Bailey's Industrial Oil and Fat Products, (3rd edition, D. Swern, ed. 1979), which is incorporated by reference herein. Fatty alcohols included among those useful herein are disclosed in the following documents, all incorporated by reference herein: U.S. Pat. No. 3,155,591; U.S. Pat. No. 4,165,369; U.S. Pat. No. 4,269,824; British Specification No. 1,532,585; and Fukushima et al., “The Effect of Cetostearyl Alcohol in Cosmetic Emulsions”, 98, Cosmetics & Toiletries, 89-112 (1983). Fatty esters included among those useful herein are disclosed in U.S. Pat. No. 3,341,465, which is also incorporated by reference herein. If included in any of the formulations according to some embodiments of the present invention, the lipid vehicle material is present at from about 0.1 weight percents to about 10.0 weight percents.

Exemplary esters for use herein include cetyl palmitate and glycerylmonostearate. Cetyl alcohol and stearyl alcohol are highly useful alcohols. A particularly useful lipid vehicle material comprises a mixture of cetyl alcohol and stearyl alcohol containing from about 55 weight percents to about 65 weight percents of cetyl alcohol.

Other vehicles, suitable for use within the hair care formulations presented herein, in the form of, for example, tonics, mousses, gels and hairsprays. Tonics, gels and non-aerosol hairsprays utilize a solvent such as water or alcohol while mousses and aerosol hairsprays additionally utilize a propellant such as trichlorofluoromethane, dichlorodifluoromethane, dichlorotetrafluoroethane, monochlorodifluoromethane, trichlorotrifluoroethane, dimethylether, propane, n-butane and isobutane, used singly or admixed in various combinations, in addition to the liquid ovicidal/pediculicidal composition. The level of propellant can be adjusted as desired but is generally from about 3 weight percents to about 30 weight percents of the mousse composition and from about 15 weight percents to about 50 weight percents of the aerosol hairspray composition. A tonic or hairspray product having a low viscosity preferably requires an emulsifying agent to keep the film-forming carbohydrate and the oil homogeneously dispersed in solution. Examples of suitable emulsifying agents include nonionic, cationic, and anionic surfactants. Examples of such materials are described supra.

Co-surfactant systems are utilized in some of the formulations presented herein. For example, combinations of anionic and nonionic surfactants or cationic and nonionic surfactants. Generally, combinations of anionic and cationic surfactants will not provide the appropriate emulsifying benefits. If such an emulsifying agent is present, it is generally present at a level of from about 0.25 weight percents to about 7.5 weight percents of the formulation. The specific surfactant materials or combinations of surfactant materials to be used and the particular levels of use are determined by those that will allow the formation of microemulsions or microsuspensions of the film-forming carbohydrate and the oil in the formulation. A non-limiting example of such a surfactant emulsifier system is the combination of lauramine oxide and cocamide DEA.

Hairspray formulations according to embodiments of the invention are particularly challenging to formulate because the film-forming carbohydrate may not be soluble in typical hairspray solvents, such as ethanol. Hence, the film-forming carbohydrate may tend to precipitate out of solution over time and form a solid mass at the bottom of the container. This solid mass is un-redispersible in the solvent upon agitation. Use of the above described surfactant emulsifying agents provides one solution to this problem. Selection of appropriate amount and type of emulsifying agents will enable the formation of a stable microemulsion of the film-forming carbohydrate and the oil in the hairspray composition. With such a method the film-forming carbohydrate and the oil does not phase-separate, and the problems of dispersion/agglomeration are substantially avoided.

Formulations as described herein in the form of a hairspray may comprise a volatile carrier system. This may comprise any of those conventionally used in resin hairspray formulations, preferably a C₁₋₆ alkanols, such as ethanol. This component “carries” the film-forming carbohydrate and the oil to the hair and than volatilizes, leaving the ovicidal/pediculicidal composition behind on the hair to provide eradicating activity against ectoparasites, as well as hairstyling hold. The carrier is present in the hairspray formulation at from about 20 weight percents to about 90 weight percents, or from about 35 weight percents to about 90 weight percents of the formulation. Water can also be used to substitute for part of the volatile carrier component.

Hairspray compositions according to some embodiments of the invention may comprise from about 20 weight percents to about 40 weight percents of the presently disclosed liquid ovicidal/pediculicidal composition having a viscosity of from about 1,000,000 centistoke to about 20,000,000 centistoke; and a volatile carrier.

An additional component, that is widely used in the hairspray formulations and in some embodiments of the present invention, is a hair setting polymer, also known as hair hold resins. Any polymer soluble or dispersible in the volatile carrier or solvent phase may be used. Solubility/dispersibility is determined at ambient conditions (e.g., temperature about 25° C. and atmospheric pressure). Suitable types of polymers include anionic, nonionic, amphoteric and cationic. Specific polymers include polyvinylpyrrolidone (PVP), copolymers of (PVP) and methylmethacrylate, copolymers of PVP and vinylacetate (VA), polyvinyl alcohol (PVA), copolymers of PVA and crotonic acid, copolymers of PVA and maleic anhydride, hydroxypropyl cellulose, hydroxypropyl guar gum, sodium polystyrene sulfonate, PVP/ethylmethacrylate/methacrylic acid terpolymer, vinyl acetate/crotonic acid/vinyl neodecanoate copolymer, octylacrylamide/acrylates copolymer, monoethyl ester of poly(methyl vinyl ether maleic acid), and octylacrylamide/acrylate/butylaminoethyl methacrylate copolymers. PVP and PVP copolymers with other monomers may also be used, as well as any mixtures thereof.

These polymers are used at a level of from about 0.25 weight percents to about 20 weight percents, or from about 1 weight percents to about 20 weight percents of the total formulation. The mass average molecular weight of the polymer is not critical, but is generally in the range of from about 2,000 to about 2,000,000.

With certain of these polymers it may be necessary to neutralize some acidic groups to promote solubility/dispersibility (e.g., PVA/crotonic acid). Examples of suitable neutralizing agents include 2-amino-2-methyl-1,3-propanediol (AMPD); 2-amino-2-ethyl-1,3 propanediol (AEPD); 2-amino-2-methyl-1-propanol (AMP); 2-amino-1-butanol (AB); monoethanolamine (MEA); diethanolamine (DEA); triethanolamine (TEA); monoisopropanolamine (MIPA); diisopropanol amine (DIPA); triisopropanolamine (TIPA); and dimethyl stearamine (DMS).

The hair care formulations described herein can contain a variety of other optional components suitable for rendering such compositions more acceptable. Such conventional optional ingredients are well known to those skilled in the art, e.g., fragrances; deodorant agents; pearlescent aids such as ethylene glycol distearate; preservatives such as benzyl alcohol, methyl paraben, propyl paraben and imidazolidinyl urea; thickeners and viscosity modifiers such as a diethanolamide of a long chain fatty acid (e.g., PEG 3 lauric diethanolamide), cocomonoethanol amide, dimethicone copolyols, guar gum and derivatives thereof, methyl cellulose, starches and derivatives thereof; fatty alcohols such as cetearyl alcohol; sodium chloride; sodium sulfate; polyvinyl alcohol; ethyl alcohol; pH adjusting agents such as citric acid, sodium citrate, succinic acid, phosphoric acid, sodium hydroxide, sodium carbonate, etc.; colorants such as any of the FD&C or D&C dyes hair oxidizing (bleaching) agents such as hydrogen peroxide, perborate and persulfate salts; hair reducing agents such as the thioglycolates; fragrances and perfumes; sequestering agents such as disodium ethylenediamine tetra-acetate; and polymer plasticizing agents such as glycerin and propylene glycol.

Such optional ingredients generally are used individually at a level of from about 0.01 weight percents to about 10.0 weight percents, or from about 0.05 weight percents to about 5.0 weight percents of the formulation.

The content of the film-forming carbohydrate and the content of the oil in any of the hair care formulations according to embodiments of the invention, can be calculated from the contents thereof in the ovicidal/pediculicidal composition presented herein, and the content of that ovicidal/pediculicidal composition in the given hair care formulation. For example, if the content of the film-forming carbohydrate is 20 weight percents in the ovicidal/pediculicidal composition, and the content of the oil is 2 weight percents in the ovicidal/pediculicidal composition, and the content of the ovicidal/pediculicidal composition in the hair care formulation is 50 weight percents, the content of the film-forming carbohydrate is 10 weight percents in the hair care formulation, and the content of the oil is 1 weight percents in the hair care formulation.

In some embodiments, when the hair care formulations according to embodiments of the invention are formulated into shampoos, the respective level of the film-forming carbohydrate incorporated therein is from about 10 weight percents to about 30 weight percents and the level of the oil is from about 1 weight percents to about 5 weight percents. In some embodiments, for a hair conditioning product the respective level of the film-forming carbohydrate incorporated therein is from about 10 weight percents to about 30 weight percents and the level of the oil is from about 2 weight percents to about 10 weight percents. In some embodiments, for hair tonic the respective level of the film-forming carbohydrate incorporated therein is from about 10 weight percents to about 30 weight percents and the level of the oil is from about 5 weight percents to about 15 weight percents.

In some embodiments, the pH of the formulations described herein is between about 3 and about 8, or between about 5 and about 8, as typically maintained for hair care products known in the art.

Also useful in the hair care formulations of the present invention are certain volatile hydrocarbons. These hydrocarbons may be either branched or unbranched, and contain from 6 to about 20 carbon atoms or from about 12 to about 16 carbon atoms.

As with all hair care formulations, the compositions and/or formulations described herein do not contain components which unduly interfere with the desired performance and physical and physicochemical characteristics of the hair care formulations.

In some embodiments, any of the hair care formulations described herein is packaged in a packaging material and is identified in print, in or on the packaging material, for use in eradicating insect ectoparasites in general, and human head lice in particular.

According to some embodiments of the present invention, the ovicidal/pediculicidal composition and a formulation of a standard hair care product, may be packaged separately. In those embodiments, the user adds the ovicidal/pediculicidal composition into the standard hair care product and uses the resulting formulation as indicated. Optionally, the user is instructed to shake (e.g., by vigorous shaking) the combined formulation.

According to another aspect of the present invention, there is provided a process for preparing the hair care formulations presented herein, which include mixing the film-forming carbohydrate, the oil and the water until a homogeneous liquid ovicidal/pediculicidal composition is obtained; and adding this liquid ovicidal/pediculicidal composition into a precursor formulation of a hair care formulation, so as to amount to a relative content from about 10 or 20 weight percents to about 60 weight percents of the total weight of the finished hair care formulation.

The hair care formulations described herein can be made using any conventional formulations and mixing techniques. In some embodiments, where a stable emulsion form is more suitable for commercial or practical reasons, the film-forming carbohydrate aqueous solution and the oil are combined with the other hair care formulation components in such a fashion so as to obtain a stable emulsion. A stable emulation can be afforded, for example, by the addition of an effective amount of an emulsifier and a suitable mechanic mixing technique which is capable of a vigorous agitation of the formulation so as to arrive at a stable emulsion thereof.

The relative amount of each of the ingredients, other than the film-forming carbohydrate and the optional oil, is determined according to the type of hair care product afforded by the formulation, which can be selected from the group consisting of a shampoo, a conditioner, a mask, a mousse, a gel, a hair spray, a lotion, a moisturizer, a tonic and a serum, and the rest of the ingredients present in the formulations are selected suitable per the particular intended product.

The hair care formulations of the present invention can be used in conventional ways to provide the hair conditioning/styling/holding as other hair care products in the market. Such ways of use depends upon the type of formulation/product employed, and generally involves application of an effective amount of the formulation to the hair, which may then be rinsed from the hair (as in the case of shampoos and some conditioning products) or allowed to remain on the hair (as in the case of spray, mousse, gel, and tonic products).

However, since the present formulations are provided as a measure against ectoparasites, the effective amount and mode of use are regarded as part of the anti-phthirapteral treatment. By “effective amount” is meant an amount sufficient to provide the desired anti-phthirapteral effect considering the density, length and texture of the hair, and the type of product used.

The formulation is applied to wet or damp hair prior to combing, drying and styling of the hair. Use of the formulations described herein in this manner provides optimal eradication of the ectoparasite in the hair. After the formulation is applied to the hair, it is maintained in the hair for a time period that ranges from 10 seconds to 10 minutes, the hair is then combed with a dense comb, dried and styled in the usual ways of the user.

Hence, according to another aspect of the present invention, there is provided a method of eradicating a population of at least one ectoparasite in human hair, as defined herein, which is effected by:

(a) applying an ovicidal/pediculicidal effective amount of the hair care formulation presented herein onto hair, wet hair as an example;

(b) leaving the formulation on the hair and scalp for a pre-determined time period; and

(c) rinsing the formulation from the hair.

According to some embodiments of the present invention, the pre-determined time period ranges from 10 seconds minute to 20 minutes, from 10 seconds minute to 10 minutes, from 10 seconds to 5 minutes, from 10 seconds to 3 minutes, from 10 seconds to 2 minutes, from 10 seconds to one minute, from 30 seconds to 1 minute, or from 1 minute to 6 minutes, from 2 minutes to 6 minutes, from 3 minutes to 6 minutes, or from 4 minutes to 6 minutes.

According to some embodiments of the present invention, the ovicidal/pediculicidal effective amount is as defined herein for “effective amount”, and can be determined by a person skilled in the art based on the hair area to be treated, the concentration of the active ingredient(s) in the formulation, the age of the subject being treated, the level of infestation, and other parameters. In some embodiments, the effective amount ranges from 10 grams to 30 grams of the hair care formulation.

According to some embodiments of the present invention, the method is also effected by, subsequent to the application of the formulation, working the formulation through the hair and scalp.

It is noted herein that the anti-ectoparasite ingredient, namely the liquid ovicidal/pediculicidal composition presented herein, may also be used directly on the hair as a hair care formulation per-se.

The use of the hair care formulations presented herein should be followed by mechanical removal of the dead or dislocated individual insects from the hair. Such a act is effectively afforded and effected by use of a dense comb, typically known as a “lice comb”. This act will remove the part of the population of ectoparasites, the hair grip thereof was loosened by the combination of film-forming carbohydrate and the oil.

Hence, in some embodiments, the method is further effected by combing the hair with a dense comb, subsequent to washing the formulation.

According to some embodiments of the invention, any of the formulations and compositions for the eradication of ectoparasites described herein can be supplemented with at least one other anti-phthiraptera agent known in the art, such as organochlorines (for example, lindane or DDT), organophosphates (for example, malathion), carbamates (for example, carbaryl), pyrethrins (for example, pyrethrum), pyrethroids (for example, permethrin, phenothrin and bio-allethrin) and protease enzymes, as well as aniseed, coconut, neem and tea tree oils, rosmary oil and other oils considered as anti-phthirapteral, and silicon oil (e.g., dimethicone). The compositions and formulations described herein can be utilized in combination with hair combing, using a particularly designed lice comb, as mentioned hereinabove, and/or in combination with any other device, such as, for example, devices that blow hot air.

It is noted that the application of the presently disclosed hair care formulations should be repeated at least twice in cases where the ectoparasite infestation is severe or lasts long enough to allow the laying of ova (eggs) in the infested hair. The repeated application of the formulations will ensure that the newly hatched nymphs will not grow and mature so as to erect another generation.

It is also noted herein that the use of the hair care formulations presented herein should be repeated after each case of re-infestation by contact with an external source of parasites, particularly in cases where the treated subject comes in social contact with other subjects suspected of being infested with the ectoparasite.

It is further noted herein that any of the formulations, compositions and methods described herein can be utilized also for preventing lice infestation in cases where such infestation is likely to occur, as long as the formulations and/or compositions are applied regularly, as long as exposure to infestation is optional.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Additional objects, advantages, and novel features of the present invention will become apparent to one ordinarily skilled in the art upon examination of the following examples, which are not intended to be limiting. Additionally, various embodiments and aspects of the present invention as delineated hereinabove and as claimed in the claims section below finds experimental support in the following examples.

### EXAMPLES

Reference is now made to the following examples, which together with the above descriptions, illustrate some embodiments of the invention in a non limiting fashion.

Although the present invention is demonstrated in ways of compositions and methods useful in treating lice and lice eggs in humans and animals, it should be understood that the compositions and methods according to some embodiments presented herein are not limited to the treatment of lice and lice eggs, rather the compositions and methods can be useful in treating other parasites and pests.

### Example 1

### A Shampoo Formulation for Eradicating Head Lice

Insecticide-based anti-lice formulations are typically tested by a lice killing rate standard. This standard, however, fails to address the recurrence of the infestation of the subject's hair by lice, which is one of the main symptoms that accompany lice infestation. The efficacy of the formulations presented herein is determined by the lack of reoccurrence of the infestation of the subject's hair by lice.

As demonstrated hereinbelow, a shampoo product was prepared and applied on more than 50 lice-infested children of both sexes, ages ranging from 4-14, living in normal conditions including social interactions in daycare and schools facilities as well as homes with other family members.

Shampoo formulation utilized glucose syrup 80/42 (Gal'am Industries Ltd., Israel, Cat No. GL1160100) as a film-forming carbohydrate, and canola oil and tocopheryl acetate as oil. The formulation is presented in Table 2 below.

TABLE 2

| | Content | |
|---|---|---|
| Ingredients | (% wt) | Function |
| Glucose | 20 | Film-forming carbohydrate |
| Canola Oil | 1 | Oil |
| Tocopheryl Acetate | 0.03 | Oil, Vitamin, Antioxidant, |
| (vitamin E acetate) | | skin conditioning agent |
| Sodium Coceth Sulfate | 11.5 | Surfactant-cleansing agent; |
| | | and emulsifying Agent |
| Cocamidopropyl Betaine | 3.8 | Antistatic agent, |
| | | hair-conditioning agent, |
| | | skin-conditioning |
| | | agent, surfactant, |
| | | emulsifier, cleansing agent |
| Perfume | 0.4 | Fragrance |
| Sodium Chloride | 0.3 | Ionic agent, Humectant |
| Sea Salt (Mans Sal) | 0.25 | Skin conditioning agent, |
| | | Ionic agent, Humectant |
| Simondsia Chinensis | 0.15 | Botanical |
| (Jojoba) Seed Extract | | |
| Verbena Officinalis | 0.1 | Fragrance, Skin |
| Leaf Extract | | conditioning agent, |
| | | Botanical |
| Anthemis Nobilis | 0.03 | Fragrance, Skin |
| (Chamomile) Extract | | conditioning agent, |
| | | Botanical |
| Triclosan | 0.04 | Preservative |
| Methylchloroisothiazolinone | 0.0009 | Preservative |
| Methylisothiazolinone | 0.0003 | Preservative |
| Water | QS to 100 | Carrier/Solvent |

All tested children were shampooed with the above formulation using standard techniques, holding the hair with the shampoo formulation for five minutes prior to rinsing. Thereafter hair was combed with a dense comb.

The results after a single use were uniform across the entire population of tested children. In all cases, the lice appeared to immerge out of the hair during the work-in of the formulation, and appeared to be dead instantly. Combing the hair in a standard manner produced a plurality of nymphs and adult lice, all appearing to be dead. Subsequent inspection of the children's hair revealed no lice or signs of lice in the hair.

The hair of the children was shampooed once daily for two days with the exemplary formulation presented in Table 2 hereinabove, but no visible signs of lice were observed. Subsequent inspection of the children's hair for the following weeks revealed no lice or signs of lice in the hair, even with children with known re-infestations and infected social environment (infested friends in kindergarten).

Children are exceptionally susceptible to infestation by phthiraptera due to their social habits (close physical contact with other children) and possibly their hormonal and secretion composition. In cases where an infestation was suspected to occur, children were treated with the exemplary formulation presented in Table 2, with no signs of lice before or after use.

### Example 2

### Exemplary Hair Care Formulations for Eradicating Head Lice—General Approach

The hair care formulations of the present embodiments can be made using any conventional formulations and mixing techniques. However, it is noted that in some embodiments it is best to form a stable emulsion from the film-forming carbohydrate aqueous solution and the oil prior to combination with the other hair care composition components.

If a volatile carrier is used in the formulations described herein to lower the viscosity of liquid ovicidal/pediculicidal composition, it may be combined with the liquid ovicidal/pediculicidal composition in several steps. Mixing the volatile carrier and the liquid ovicidal/pediculicidal composition together this way avoids disruption of the homogeneous emulsion of the liquid ovicidal/pediculicidal composition. If a resin is also used in the formulation, it may be mixed with the volatile carrier prior to combination of the carrier material with the liquid ovicidal/pediculicidal composition. The resulting mixture can then be formulated into a variety of hair care products including tonics, shampoos, conditioners, mousses, gels and hair sprays. It is noted that the pH of the final formulation is adjusted, if necessary, to be between 3 and 7, preferably between 6 and 7.

Methods of making various types of hair care compositions are described more specifically in the following examples.

### Example 3

### Liquid Ovicidal/Pediculicidal Composition Premix

Glucose syrup (DE 43) as a film-forming carbohydrate—40 weight percents;

Canola oil as oil—10 weight percents; and

Deionized water—70 weight percents (QS, Quantum Sufficiat to complete to 100 weight percents).

Using a mixer such as a ribbon type blender, the components of the liquid ovicidal/pediculicidal composition premix are combined and mixed until a homogeneous emulsion is formed.

### Example 4

### Shampoo for Eradicating Head Lice

The ingredients and their amounts in weight percentages for two optional shampoo formulations A and B, are presented in Table 3 below.

TABLE 3

| Ingredient | A | B |
|---|---|---|
| Ovicidal/pediculicidal composition premix | 30 | 50 |
| (see, Example 3 hereinabove) | | |
| Ammonium Lauryl Sulfate | 13.5 | 13.5 |
| Ammonium Laureth Sulfate | 4 | 4 |
| Ammonium Xylene Sulfonate | 0.1 | 0.1 |
| Dimethicone Gum | 0.16 | 0.8 |
| Octylacrylamide/acrylate/butyl | 0.04 | 0.02 |
| aminoethyl methacrylate copolymer | | |
| Preservative | 0.033 | 0.033 |
| Cocoamide MEA | 4 | 4 |
| Ethylene Glycol Distearate | 2 | 2 |
| Cetearyl Alcohol | 0.6 | 1 |
| Sodium Citrate | 0.05 | 0.05 |
| Citric Acid | 0.05 | 0.05 |
| Sodium Hydroxide | 0.01 | 0.01 |
| Sodium Chloride | 1 | 1 |
| Colorant | QS | QS |
| Deionized water | QS | QS |

The shampoo processing is effected as follows: ammonium lauryl sulfate, citric acid, sodium citrate and sodium hydroxide are added to the distilled water at about 15° C. The mixture is heated to from 70° C. to 80° C. The cocamide MEA and glycol distearate are added at this point. The ammonium laureth-3 sulfate, cetearyl alcohol and the ovicidal/pediculicidal composition premix are blended at 70° C. to 90° C. This mixture is added to the batch following the glycol distearate. The preservative and fragrance are added thereafter. The batch is mixed for 5 minutes and then cooled to room temperature (15° C. to 25° C.). Sodium chloride and ammonium xylene sulfonate are added for viscosity control as needed. The final compositions have a pH of from about 5.0 to about 6.0.

These compositions are used in the same way one would use a standard shampoo, all but keeping the shampoo on the hair for 4-10 minutes. The hair is then combed with a dense comb, dried and styled in the usual way.

### Example 5

### Conditioner for Eradicating Head Lice

The ingredients and their amounts in weight percentages for two optional conditioner formulations A and B, are presented in Table 4 below.

TABLE 4

| Ingredient | A | B |
|---|---|---|
| Ovicidal/pediculicidal composition premix | 30 | 50 |
| (Example 3 hereinabove) | | |
| Cetyl Alcohol | 1 | 1 |
| Quaternium 18 | 0.85 | 0.85 |
| Hydroxyethyl Cellulose | 0.5 | 0.5 |
| Stearimidopropyl Dimethylamine | 0.5 | 0.5 |
| Ceteareth-20 | 0.35 | 0.35 |
| Fragrance | 0.25 | 0.25 |
| Citric Acid | 0.13 | 0.13 |
| Dimethicone Copolyol | 0.1 | 0.1 |
| Octylacrylamide/acrylate/butyl | 0.04 | 0.1 |
| aminoethyl methacrylate copolymer | | |
| Preservative | 0.033 | 0.033 |
| Colorant | QS | QS |
| Deionized water | QS | QS |

The conditioner processing is effected as follows: hydroxyethyl cellulose is added to the distilled water at a temperature of 15° C. to 40° C. This mixture is well-dispersed, then heated to a temperature of from 60° C. to 90° C. Ingredients 2 through 8 are added to the batch while the temperature is maintained in this range. The mixture is stirred for approximately 10 minutes, then cooled to approximately 50° C. The remaining ingredients and the ovicidal/pediculicidal composition premix are added at this temperature, then cooled to room temperature. The finished compositions have a pH of from about 3.5 to about 4.5.

These conditioner formulations are used as one would use standard rinse-type conditioning products, i.e., after shampooing, the conditioner is applied to the hair, allowed to stay on the hair for at least 4-10 minutes, and then rinsed from the hair. The hair is then combed with a dense comb, dried and styled in the usual way.

### Example 6

### Non-Aerosol Hair Tonic Spray for Eradicating Head Lice

Ovicidal/pediculicidal composition premix (see, Example 3)—20 weight percents;

PVP/VA Copolymer—2 weight percents; Lauramine Oxide—1 weight percents;

Cocamide DEA—0.8 weight percents;

Carbomer 956—0.2 weight percents;

Potassium Hydroxide QS to adjust pH to between 6 and 7

Fragrance—QS to taste;

Preservative—QS; and

Water—QS to complete 100 weight percents.

A non-aerosol hair tonic spray product is prepared as follows. The lauramide oxide is mixed with part of the water at a ratio of 4 to 1 with, for example, a ribbon type mixer until homogeneous. The cocamide DEA is added and mixed until homogeneous. The ovicidal/pediculicidal composition premix prepared as presented hereinabove is added and mixed until homogeneous.

The remainder of the water is put into a stainless steel mixing vessel. The Carbomer 956 is mixed into the water using, for example, a triblender or eductor mixer. Mixing is continued until the Carbomer is completely dissolved. The potassium hydroxide is added while mixing.

The premix is added while mixing until homogeneous. The PVP/VA is then added and mixing is continued until the batch is homogeneous. The preservative is added and mixing is continued until homogeneous. The perfume is added and mixing is continued for an additional 10 minutes. Once the batch is well-mixed homogenization of the batch is performed using conventional apparatus. The final product is an opaque liquid having a pH of about 6 and 7.

This exemplary hair tonic is sprayed onto damp hair and the hair is then combed, styled and dried. The amount of tonic used will depend on the volume/hold benefits desired and the amount of hair being treated as well as the texture of the hair.

### Example 7

### Mousse for Eradicating Head Lice

The ingredients and their amounts in weight percentages for two optional hair mousse formulations A and B, are presented in Table 5 below.

TABLE 5

| Ingredient | A | B |
|---|---|---|
| Ovicidal/pediculicidal composition premix | 20 | 30 |
| A-46 propellant | 7.5 | 7.5 |
| PVP/VA Copolymer (50% active) | 1 | 2.5 |
| Lauramine Oxide | 1 | 1 |
| Cocamide DEA | 0.8 | 0.8 |
| Fragrance | QS | QS |
| Preservative | QS | QS |
| Deionized water | QS | QS |

The exemplary aerosol mousse according to some embodiments of the present invention, are prepared by combining all ingredients except the aerosol propellant into a batch called the concentrate. This concentrate is made by combining with agitation all of the ingredients except for the preservative and the premix prepared as described hereinabove, and mixing continued until these are thoroughly dispersed. The resulting mixture is then homogenized using conventional apparatus. The resulting concentrate has a pH of from 6 to 7. Aerosol mousse cans are prepared by placing 135 grams of concentrate into 5 ounce aluminum epoxy lined cans, placing mousse valves on can tops, drawing a vacuum to evacuate can headspace (to remove air), and crimping the valves into place. The propellant (15 grams) is added by pressure filling through the valve stem.

These formulations are massaged into clean/damp hair and the hair is then kept for 4-10 minutes, dried and styled. The amount of mousse used will depend on the volume/hold benefits desired and the amount of hair being treated as well as the texture of the hair.

### Example 8

### Gel for Eradicating Head Lice

The ingredients and their amounts in weight percentages for two optional hair gel formulations A and B, are presented in Table 6 below.

TABLE 6

| Ingredient | A | B |
|---|---|---|
| Ovicidal/pediculicidal composition premix | 30 | 50 |
| (Example 3 hereinabove) | | |
| PVP/VA Copolymer | 1 | 2.5 |
| Lauramine Oxide | 1 | 1 |
| Cocamide DEA | 0.8 | 0.8 |
| Carbomer 940 | 0.4 | 0.6 |
| Triethanolamine | 0.36 | 0.56 |
| Octylacrylamide/acrylate/butyl | 0.01 | 0.1 |
| aminoethyl methacrylate copolymer | | |
| Colorant | QS | QS |
| Fragrance | 0.25 | 0.25 |
| Preservative | 0.033 | 0.033 |
| Deionized water | QS | QS |

The gel formulations according to some embodiments of the present invention are prepared using the method outlined in the example for the hair tonic, except that the Carbomer 940 is substituted for the Carbomer 956 and the triethanolamine is added before the preservative and mixed in until homogeneous. These compositions have a pH of about 6 to 7.

These gel formulations are used in the same way as the mousse compositions described hereinabove.

### Example 9

### Non-Aerosol Hairspray for Eradicating Head Lice

The ingredients and their amounts in weight percentages for an optional Non-aerosol hairspray formulation is presented below.

Ovicidal/pediculicidal composition premix (see, Example 3)—20 weight percents;

PVP/VA copolymer (50/50)—10 weight percents;

Tixogel VP—0.10 weight percents;

Polydimethysiloxane gum—0.2 weight percents;

Octyl Salicylate 0.01 weight percents;

Keratin AMino Acids—0.001 weight percents;

Fragrance—0.10 weight percents;

Ethanol (190 proof)—QS to 100 weight percents;

The Tixogel® is added to the ovicidal/pediculicidal composition premix and mixed until homogeneous, and the ethanol is thereafter added and mixed until homogeneous. Using conventional mixing the PVP/VA/copolymer is added. The octyl salicylate, keratin amino acids, and fragrance are mixed into the composition in that order.

### Example 10

### Clinical Studies

The efficacy of a hair care formulation as described, for example, in Example 1 hereinabove, is tested in a double blind placebo control trial.

Sixty (60) healthy children, aged 6-12 years, are tested and documented for the presence of hair lice a day before trial onset and are randomly divided into two groups, each being of 30 children. Children in Group I are treated with the tested formulation and children I Group II are treated with the formulation of the same hair product, devoid of the ovicidal/pediculicidal composition. Treatment includes a daily hair wash, effected by applying the formulation to wet hair, rubbing the hair and scalp, maintaining the formulation on the hair and scalp for 3 minutes and rinsing the formulation in water. Hair washing is followed by combing the hair during 5 minutes with a dense comb.

Treatment is continued for 10 days, under daily supervision.

At the end of the 10-days period, the children in the two groups are tested again for the presence of lice, and documented accordingly.

Following an additional month, treatment crossover is effected: Group II, which previously was treated without the composition, is not treated with a formulation as described herein, while Group I, previously treated with the composition, is now treated with the placebo.

The same protocol as described hereinabove is applied, and same documentation is performed.

At the end of the second treatment period, results are analyzed for determining a statistically significant improved effect of the tested formulation, as compared with the placebo.

For all the examples presented hereinabove and in other embodiments of the present invention, substantially similar results are obtained when an equivalent amount of any one material sold under one trade name, is substituted with a similar material sold under another trade name.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A hair care formulation comprising from about 20 weight percents to about 60 weight percents of a liquid ovicidal/pediculicidal composition, said liquid ovicidal/pediculicidal composition comprising: a film-forming carbohydrate in an amount that ranges from about 5 weight percents to about 50 weight percents; and
water.

2. The hair care formulation of claim 1, being identified for eradicating a population of at least one ectoparasite.

3. The hair care formulation of claim 1, being washable within a time period of less than 5 minutes from hair at a length of 5 cm under a water shower stream of 20 liters per minutes and a water temperature of 37° C.

4. (canceled)

5. The hair care formulation of claim 1, wherein said composition further comprises oil in an amount that ranges from 1 weight percent tot 20 weight percents of the total weight of said composition.

6. The hair care formulation of claim 5, wherein said composition further comprises an emulsifier in an amount that ranges from 0.5 weight percent to 5 weight percents of the total weight of said composition.

7-8. (canceled)

9. The hair care formulation of claim 1, wherein said ectoparasite is Pediculus humanus capitis.

10. The hair care formulation of claim 1, wherein said film-forming carbohydrate is selected from the group consisting of a monosaccharide, a disaccharide, a polysaccharide, a purified saccharide, an impure saccharide, a crude saccharide, a mixture of saccharides, molasses, a syrup, a treacle and any combination thereof.

11. The hair care formulation of claim 10, wherein said film-forming carbohydrate comprises at least one substance selected from the group consisting of allose, altrose, arabinose, a beet-sugar syrup, a cane-sugar syrup, a corn syrup, cellobiose, deoxyribose, dextrose, erythrose, erythrulose, fructose, fructose syrup, fucose, fructan, galactitol, galactosamine, galactose, gentiobiose, gluconic acid, glucosamine, glucose, glucose syrup, glucuronic acid, glycerol (glycerine), gulose, a high fructose starch-based syrup (HFSS), a high-fructose corn syrup (HFCS), idose, inositol, isomalt, isoglucose, isomaltose, isomaltulose/palatinose, lactitol, lactose, lactulose, levulose, lyxose, maltitol, maltose, maltodextrin, maltotriose, mannitol, mannosamine, mannose, melibiose, nystose, propylene glycol, psicose, raffinose, rhamnose, ribose, ribulose, saccharose, sorbitol, sorbose, sucrose, tagatose, talose, threose, trehalose, turanose, xylitol, xylobiose, xylose, xylulose, α,β-trehalose (neotrehalose), β,β-trehalose (isotrehalose), β-allopyranose, and mixtures and syrups thereof.

12. The hair care formulation of claim 11, wherein said film-forming carbohydrate comprises glucose syrup having a dextrose equivalent value (DE) lower than 55.

13. (canceled)

14. The hair care formulation of claim 11, wherein said film-forming carbohydrate is glucose syrup having a dextrose equivalent value (DE) of 42.

15-17. (canceled)

18. The hair care formulation of claim 1, being in a form of a shampoo, the formulation further comprising at least one ingredient selected from the group consisting of a detergent or surfactant, a salt, an acid and a humectant.

19. (canceled)

20. The hair care formulation of claim 1, being in a form of a conditioner, the formulation further comprising at least one ingredient selected from the group consisting of a moisturizer/humectant, an emulsifier, a reconstructor, an acid and a buffer.

21-23. (canceled)

24. A process for preparing the hair care formulation of claim 1, the process comprising:
adding said liquid ovicidal/pediculicidal composition into a formulation of a hair care composition selected from the group consisting of a shampoo, a conditioner, a mask, a gel, a lotion, a moisturizer, a tonic and a serum.

25. A method of eradicating a population of at least one ectoparasite in human hair, the method comprising:
(a) applying a disinfesting effective amount of the hair care formulation of claim 1 to wet hair;
(b) leaving said formulation on the hair and scalp for a pre-determined time period; and
(c) rinsing said formulation from the hair.

26. The method of claim 25, wherein said pre-determined time period ranges from 1 minute to 20 minutes.

27. (canceled)

28. The method of claim 26, wherein said pre-determined time period ranges from 1 minute to 6 minutes.

29. The method of claim 25, wherein said effective amount ranges from 10 grams to 30 grams of the hair care formulation.

30. The method of claim 25, further comprising, subsequent to said applying, working said formulation through the hair and scalp.

31. A liquid ovicidal/pediculicidal composition comprising:
water; and
a film-forming carbohydrate in an amount that ranges from 5 weight percents to 50 weight percents, said carbohydrate forms a film on a surface when a solution thereof is dehydrated and/or dried and/or exposed to body temperature, the composition being identified for eradicating a population of at least one ectoparasite.

32. The liquid ovicidal/pediculicidal composition of claim 31, further comprising oil in an amount that ranges from 1 weight percent to 20 weight percents of said composition.

33. The liquid ovicidal/pediculicidal composition of claim 32, further comprising an emulsifier in an amount that ranges from 0.5 weight percent to 5 weight percents.

34. (canceled)

35. The liquid ovicidal/pediculicidal composition of claim 31, wherein said ectoparasite is Pediculus humanus capitis.

36. The liquid ovicidal/pediculicidal composition of claim 31, wherein said film-forming carbohydrate is selected from the group consisting of a monosaccharide, a disaccharide, a polysaccharide, a purified saccharide, an impure saccharide, a crude saccharide, a mixture of saccharides, molasses, a syrup, a treacle and any combination thereof.

37. The liquid ovicidal/pediculicidal composition of claim 36, wherein said film-forming carbohydrate comprises at least one substance selected from the group consisting of allose, altrose, arabinose, a beet-sugar syrup, a cane-sugar syrup, a corn syrup, cellobiose, deoxyribose, dextrose, erythrose, erythrulose, fructose, fructose syrup, fucose, fructan, galactitol, galactosamine, galactose, gentiobiose, gluconic acid, glucosamine, glucose, glucose syrup, glucuronic acid, glycerol (glycerine), gulose, a high fructose starch-based syrup (HFSS), a high-fructose corn syrup (HFCS), idose, inositol, isomalt, isoglucose, isomaltose, isomaltulose/palatinose, lactitol, lactose, lactulose, levulose, lyxose, maltitol, maltose, maltodextrin, maltotriose, mannitol, mannosamine, mannose, melibiose, nystose, propylene glycol, psicose, raffinose, rhamnose, ribose, ribulose, saccharose, sorbitol, sorbose, sucrose, tagatose, talose, threose, trehalose, turanose, xylitol, xylobiose, xylose, xylulose, α,β-trehalose (neotrehalose), β,β-trehalose (isotrehalose), β-allopyranose, and mixtures and syrups thereof.

38. The liquid ovicidal/pediculicidal composition of claim 37, wherein said film-forming carbohydrate comprises glucose syrup having a dextrose equivalent value (DE) lower than 55.

39. (canceled)

40. The liquid ovicidal/pediculicidal composition of claim 37, wherein said film-forming carbohydrate is glucose syrup having a dextrose equivalent value (DE) of 42.

41-43. (canceled)

44. A process for preparing the liquid/pediculicidal composition of claim 31, the process comprising:
mixing said film-forming carbohydrate, an oil, if present, an emulsifier, if present, and said water.

45-49. (canceled)

50. The method of claim 25, wherein said hair care formulation is being washable within a time period of less than 5 minutes from hair at a length of 5 cm under a water shower stream of 20 liters per minute and a water temperature of 37° C.

51. The method of claim 25, wherein said liquid ovicidal/pediculicidal composition further comprises oil in an amount that ranges from 1 weight percent tot 20 weight percents of the total weight of said composition.

52. The method of claim 51, wherein said liquid ovicidal/pediculicidal composition further comprises an emulsifier in an amount that ranges from 0.5 weight percent to 5 weight percents of the total weight of said composition.

53. The method of claim 25, wherein said ectoparasite is Pediculus humanus capitis.

54. The method of claim 25, wherein said film-forming carbohydrate is selected from the group consisting of a monosaccharide, a disaccharide, a polysaccharide, a purified saccharide, an impure saccharide, a crude saccharide, a mixture of saccharides, molasses, a syrup, a treacle and any combination thereof.

55. The method of claim 54, wherein said film-forming carbohydrate comprises at least one substance selected from the group consisting of allose, altrose, arabinose, a beet-sugar syrup, a cane-sugar syrup, a corn syrup, cellobiose, deoxyribose, dextrose, erythrose, erythrulose, fructose, fructose syrup, fucose, fructan, galactitol, galactosamine, galactose, gentiobiose, gluconic acid, glucosamine, glucose, glucose syrup, glucuronic acid, glycerol (glycerine), gulose, a high fructose starch-based syrup (HFSS), a high-fructose corn syrup (HFCS), idose, inositol, isomalt, isoglucose, isomaltose, isomaltulose/palatinose, lactitol, lactose, lactulose, levulose, lyxose, maltitol, maltose, maltodextrin, maltotriose, mannitol, mannosamine, mannose, melibiose, nystose, propylene glycol, psicose, raffinose, rhamnose, ribose, ribulose, saccharose, sorbitol, sorbose, sucrose, tagatose, talose, threose, trehalose, turanose, xylitol, xylobiose, xylose, xylulose, α,β-trehalose (neotrehalose), β,β-trehalose (isotrehalose), β-allopyranose, and mixtures and syrups thereof.

56. The method of claim 55, wherein said film-forming carbohydrate comprises glucose syrup having a dextrose equivalent value (DE) lower than 55.

57. The method of claim 55, wherein said film-forming carbohydrate is glucose syrup having a dextrose equivalent value (DE) of 42.

58. The method of claim 25, wherein said hair care formulation is in a form of a shampoo, the formulation further comprising at least one ingredient selected from the group consisting of a detergent or surfactant, a salt, an acid and a humectant.

59. The method of claim 25, wherein said hair care formulation is in a form of a conditioner, the formulation further comprising at least one ingredient selected from the group consisting of a moisturizer/humectant, an emulsifier, a reconstructor, an acid and a buffer.
